(19) United States
(12) Patent Application Publication
(10) Pub. No.: US 2019/0322653 A1
(43) Pub. Date: Oct. 24, 2019

(54) METHODS FOR SYNTHESIZING HIGH PURITY EPOXY COMPOUNDS AND PRODUCTS OBTAINED THEREFROM

(71) Applicant: SABIC GLOBAL TECHNOLOGIES B.V., Bergen Op Zoom (NL)

(72) Inventors: Edward Norman PETERS, Selkirk, NY (US); Jaykisor PAL, Karnataka, Bangalore (IN); Mallika Salkod MESHWAR, Karnataka, Bangalore (IN); Shubashree SWAMINATHAN, Karnataka, Bangalore (IN); Gaurav MEDIRATTA, Karnataka, Bangalore (IN)

(21) Appl. No.: 16/475,759

(22) PCT Filed: Jan. 3, 2018
(86) PCT No.: PCT/US2018/012144

§ 371 Date: Jul. 3, 2019

(30) Foreign Application Priority Data
Jan. 4, 2017 (IN) 201711000314

(51) Int. Cl. C07D 405/14; C07D 407/12; B01J 31/02

(52) U.S. Cl. CPC C07D 405/14; C08G 59/28; B01J 31/0239; C07D 407/12

(57) ABSTRACT

A process for preparing a diglycidyl ether of formula (1) to (9) with epichlorohydrin and a catalyst, for a time and at a temperature to provide a first reaction mixture comprising a dichlorohydrin of formula (1-d) to (9-d); adding a base to the first reaction mixture to provide a second reaction mixture over a period of 15 minutes to 3 hours; and agitating the second reaction mixture for 2 to 5 hours at 40 to 60° C., to provide an as-synthesized diglycidyl ether of formula (1) to (9) having a purity of 96 to 99% or greater as determined by high performance liquid chromatography is provided. An as-synthesized or isolated diglycidyl ether of formula (1) to (9) made by the provided process is provided. A cured composition comprising the cured product of the provided diglycidyl ether, and an article comprising the same are provided.

### BACKGROUND

Epoxy polymers are used in a wide variety of applications, including protective coatings, adhesives, electronic laminates, flooring and paving applications, glass fiber-reinforced pipes, and automotive parts. In their cured form, epoxy polymers offer desirable properties including good adhesion to other materials, excellent resistance to corrosion and chemicals, high tensile strength, and good electrical resistance. However, the current methods to make epoxy polymers results in impurities that can require one or more additional purification steps before use.

There accordingly remains the need for methods of synthesizing high purity epoxy polymers and high purity epoxy compounds.

### SUMMARY

A process for preparing a diglycidyl ether of formula (1) to (9) wherein R^{a} and R^{b} at each occurrence are each independently halogen, C₁₋₁₂ alkyl, C₂₋₁₂ alkenyl, C₃₋₈ cycloalkyl, or C₁₋₁₂ alkoxy, p and q at each occurrence are each independently 0 to 4, R¹³ at each occurrence is independently halogen or C₁₋₆ alkyl, c at each occurrence is independently 0 to 4, R¹⁴ at each occurrence is independently C₁₋₆ alkyl, phenyl, or phenyl substituted with up to five halogens or C₁₋₆ alkyls, R^{g} at each occurrence is independently halogen or C₁₋₁₂ alkyl, or two R^{g} groups together with the carbon atoms to which they are attached form a four-, five, or six-membered cycloalkyl group, and t is 0 to 10; the process comprising reacting a bisphenol of formula (1-b) to (9-b) wherein R^{a}, R^{b}, p, q, R¹³, R¹⁴, c, R^{g}, and t are as defined in formulas (1) to (9), with epichlorohydrin and a catalyst, for a time and at a temperature to provide a first reaction mixture comprising a di-chlorohydrin of formula (1-d) to (9-d) wherein R^{a}, R^{b}, p, q, R¹³, R¹⁴, c, R^{g}, and t are as defined in formulas (1) to (9); adding a base to the first reaction mixture to provide a second reaction mixture over a period of 15 minutes to 3 hours; and agitating the second reaction mixture for 2 to 5 hours at 40 to 60° C., to provide an as-synthesized diglycidyl ether of formula (1) to (9) having a purity of 96 to 99% or greater as determined by high performance liquid chromatography is provided. An as-synthesized or isolated diglycidyl ether of formula (1) to (9) made by the provided process is provided. A cured composition comprising the cured product of the provided diglycidyl ether, and an article comprising the same are provided.

### BRIEF DESCRIPTION OF THE DRAWINGS

The following figures are exemplary embodiments.

FIGS. 1A, 1B, 1C, and 1D shows the disappearance of starting material (PPPBP) and phenolic hydroxy group-containing intermediates (PCH and PE) with time measured by HPLC area percentage.

FIG. 2 shows formation of PPPBP-DGE over time using different catalysts.

FIGS. 3A and 3B shows HPLC area percentage versus PPPBP/ECH mole ratio for the reaction with TBAB as catalyst for samples taken at 6 hours (FIG. 3A) and 7 hours (FIG. 3B).

FIG. 4 shows a plot of dimer HPLC area percentage versus PPPBP/ECH mole ratio for reactions with TBAB as catalyst for samples taken at 5 hours, 6 hours, and 7 hours.

FIGS. 5A and 5B shows HPLC area percentage of PPPBP and other reaction intermediates versus PPPBP/ECH mole ratio for the reaction with HEGCl as catalyst for samples taken 4 hours (FIG. 5A) and 7 hours (FIG. 5B).

FIG. 6 shows the plot of dimer area percentage versus PPPBP/ECH mole ratio for reactions with HEGCl as catalyst.

FIGS. 7A, 7B, and 7C show residual PPPBP (FIG. 7A), PPPBP-DGE (FIG. 7B), and dimer (FIG. 7C) concentration over time for a reaction where TBAB was used as catalyst at various levels.

FIGS. 8A, 8B, and 8C shows residual PPPBP (FIG. 8A), PPPBP-DGE (FIG. 8B), and dimer (FIG. 8C) concentration over time for a reaction where HEGCl was used as catalyst at various levels.

FIGS. 9A, 9B, and 9C shows the residual PPPBP (FIG. 9A), PPPBP-DGE (FIG. 9B), and dimer (FIG. 9C) concentration with time respectively for a reaction where reaction were carried out at 50° C. and 70° C., PPPBP/ECH ratio about 1/60 and catalyst content about 10 mol %.

FIG. 10 shows an HPLC chromatogram of the final product formed by reacting PPPBP/ECH at a mol ratio of 1/60, with 10 mol % HEGCl at 70° C., followed by addition of NaOH.

FIG. 11 shows an HPLC chromatogram of the final product formed by reacting PPPBP/ECH at a mol ratio of 1/60, with 10 mol % HEGCl at 70° C., followed by addition of NaOH using a modified stepwise process.

FIG. 12 shows the change in residual PPPBP concentration over time when different solvents are used as reaction media.

FIG. 13 shows the purity of PPPBP-DGE obtained and the amount of dimer formed after 3 hours of addition of base to the reaction mixture of Process D.

FIG. 14 shows a chromatogram of the final reaction mixture of Process E.

FIG. 15 shows the residual PPPBP, PPPBP-DGE, and dimer content with progress of reaction carried out in accordance with Process E, using a DMSO solution of PPPBP.

FIG. 16 is a ¹HNMR spectrum of PPPBP-DGE used to estimate the epoxy equivalent weight (EEW) of the isolated product.

### DETAILED DESCRIPTION

The inventors hereof have discovered a method of synthesizing high purity diglycidyl ethers of bisphenols such as 2-phenyl-3,3′-bis(4-hydroxyphenyl) phthalimidine (also known as N-phenyl phenolphthalein bisphenol (“PPPBP”)). Reaction of a bisphenol with epichlorohydrin, in presence of a base, is known to provide the corresponding diglycidyl ether. Although the chemistry is well known, the reaction product is often contaminated by a mixture of byproducts. These byproducts can lower the overall purity of the desired product. The byproducts formed during the reaction have been identified as the oligomers of diglycidyl ether and the mono- or di-chlorohydrins of the bisphenol. While the oligomers of diglycidyl ether are formed as a result of the reaction between bisphenol and diglycidyl ether, the mono- or di-chlorohydrins of bisphenol are formed as an intermediate in the reaction between bisphenol and epichlorohydrin. The presence of a base in the reaction mixture not only results in conversion of chlorohydrins to the desired diglycidyl ether, but also at the same time catalyzes oligomerization. In other words, both the desired (epoxidation) and undesired (oligomerization) reactions happen simultaneously.

The inventors hereof have discovered a method for synthesizing high purity (greater than or equal to 97% purity) diglycidyl ethers of bisphenols without requiring additional downstream purification steps such as column chromatography. The method involves reacting a bisphenol with epichlorohydrin and a catalyst, for a time and at a temperature to form a chlorohydrin intermediate. The chlorohydrin intermediate then undergoes base assisted ring closure to the corresponding diglycidyl ether. In particular, the base assisted ring closure is carried out in two steps as further described below.

The diglycidyl ethers formed by the method are compounds of formulas (1) to (9):

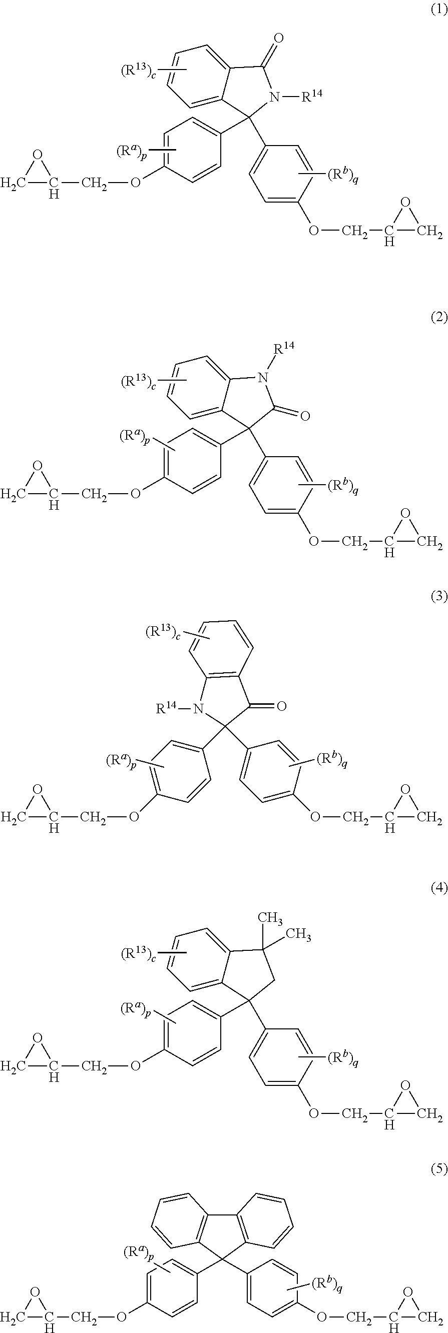

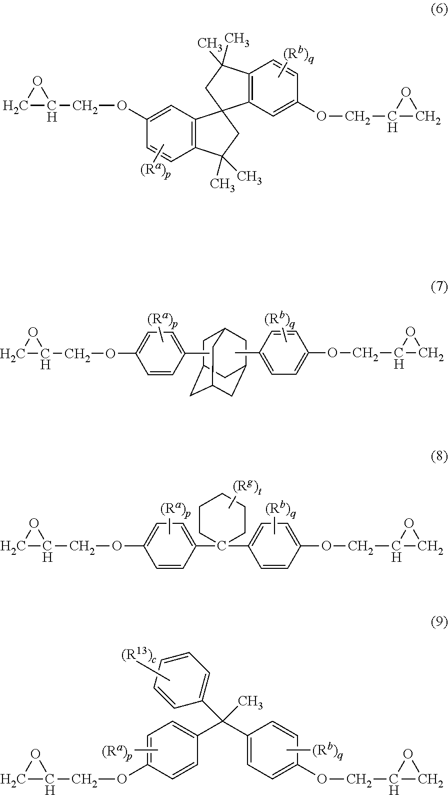

wherein R^{a} and R^{b} at each occurrence are each independently halogen, C₁₋₁₂ alkyl, C₂₋₁₂ alkenyl, C₃₋₈ cycloalkyl, or C₁₋₁₂ alkoxy; p and q at each occurrence are each independently 0 to 4; R¹³ at each occurrence is independently halogen or C₁₋₆ alkyl; c at each occurrence is independently 0 to 4; R¹⁴ at each occurrence is independently C₁₋₆ alkyl, phenyl, or phenyl substituted with up to five halogens or C₁₋₆ alkyls; R^{g} at each occurrence is independently halogen or C₁₋₁₂ alkyl, or two R^{g} groups together with the carbon atoms to which they are attached form a four-, five, or six-membered cycloalkyl group; and t is 0 to 10.

In some embodiments, R^{a} and R^{b} at each occurrence are each independently halogen, C₁₋₆ alkyl, C₂₋₆ alkenyl, C₄₋₆ cycloalkyl, or C₁₋₆ alkoxy; p and q at each occurrence are each independently 0 to 2; R¹³ at each occurrence is independently halogen or C₁₋₃ alkyl; c at each occurrence is independently 0 to 2; R¹⁴ at each occurrence is independently C₁₋₃ alkyl, phenyl, or phenyl substituted with up to two halogens or C₁₋₃ alkyls; R^{g} at each occurrence is independently halogen or C₁₋₆ alkyl, or two R^{g} groups together with the carbon atoms to which they are attached form a five or six-membered cycloalkyl group; and t is 0 to 5.

In some embodiments, R^{a} and R^{b} at each occurrence are each independently halogen, C₁₋₃ alkyl, C₂₋₄ alkenyl, or C₁₋₃ alkoxy; p and q at each occurrence are each independently 0 or 1; R¹³ at each occurrence is independently C₁₋₃ alkyl; c at each occurrence is independently 0 or 1; R¹⁴ at each occurrence is independently C₁₋₃ alkyl or phenyl; R^{g} at each occurrence is independently C₁₋₃ alkyl or two R^{g} groups together with the carbon atoms to which they are attached form a five, or six-membered cycloalkyl group; and t is 0 to 5.

In some embodiments, R^{a} and R^{b} at each occurrence are each independently C₁₋₃ alkyl; p and q at each occurrence are each independently 0 or 1; c at each occurrence is 0; R¹⁴ at each occurrence is independently methyl or phenyl; R^{g} at each occurrence is independently methyl or two R^{g} groups together with the carbon atoms to which they are attached form a five or six-membered cycloalkyl group; and t is 0 to 4.

In still another embodiment, R^{a} and R^{b} at each occurrence are each methyl; p and q at each occurrence are each independently 0 or 1; c is zero; and R¹⁴ at each occurrence is independently a phenyl, or phenyl substituted with up to two methyl groups.

The diglycidyl ether compounds can have formula (1-a), (2-a), (3-a), (4-a), (5-a), (6-a), (7-a), (8-a), (8-a′), (8-a″), or (9-a).

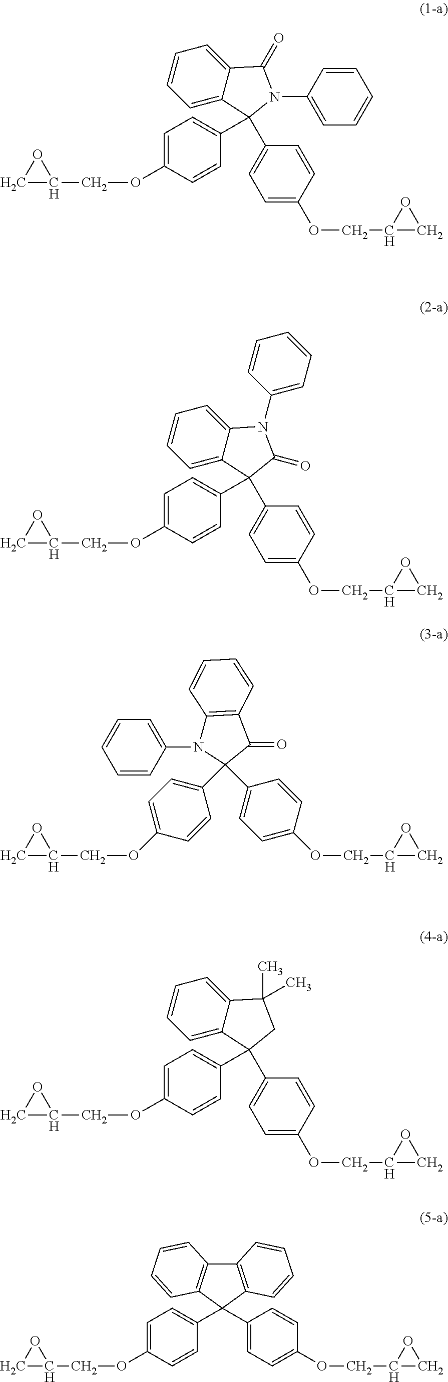 [SMILES: O=C1c2ccccc2C(c2ccc(OCC3CO3)cc2)(c2ccc(OCC3CO3)cc2)N1c1ccccc1 ; O=C1N(c2ccccc2)c2ccccc2C1(c1ccc(OCC2CO2)cc1)c1ccc(OCC2CO2)cc1 ; O=C1c2ccccc2N(c2ccccc2)C1(c1ccc(OCC2CO2)cc1)c1ccc(OCC2CO2)cc1 ; CC1(C)CC(c2ccc(OCC3CO3)cc2)(c2ccc(OCC3CO3)cc2)c2ccccc21 ; c1ccc2c(c1)-c1ccccc1C2(c1ccc(OCC2CO2)cc1)c1ccc(OCC2CO2)cc1]

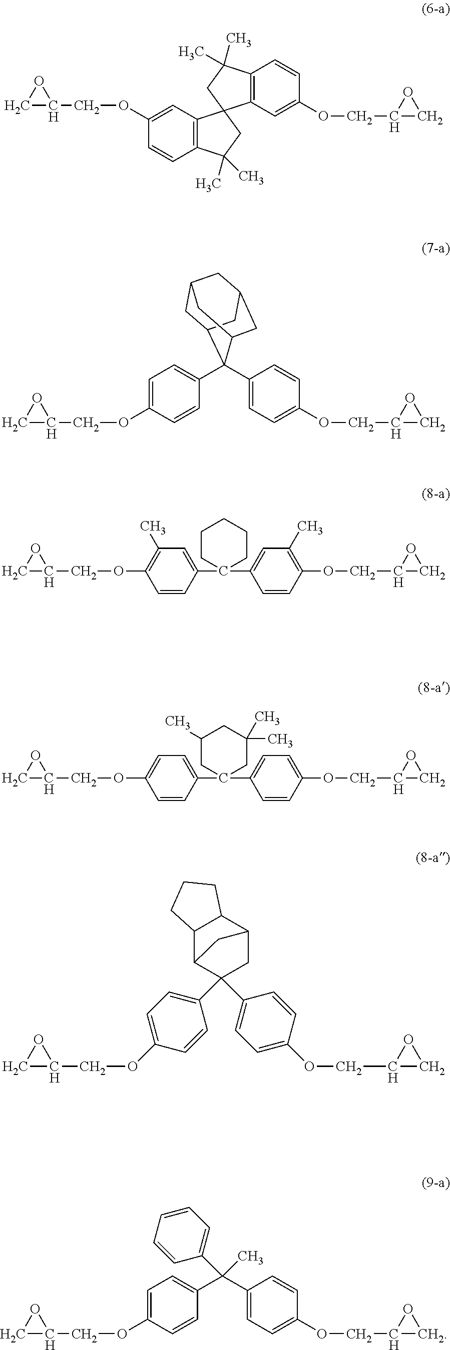 [SMILES: CC1(C)CC2(CC(C)(C)c3ccc(OCC4CO4)cc32)c2cc(OCC3CO3)ccc21 ; c1cc(C2(c3ccc(OCC4CO4)cc3)C3CC4CC(C3)CC2C4)ccc1OCC1CO1 ; Cc1cc(C2(c3ccc(OCC4CO4)c(C)c3)CCCCC2)ccc1OCC1CO1 ; CC1CC(C)(C)CC(c2ccc(OCC3CO3)cc2)(c2ccc(OCC3CO3)cc2)C1 ; c1cc(C2(c3ccc(OCC4CO4)cc3)CC3CC2C2CCCC32)ccc1OCC1CO1 ; CC(c1ccccc1)(c1ccc(OCC2CO2)cc1)c1ccc(OCC2CO2)cc1]

In a specific embodiment, the diglycidyl ether is a diglycidyl ether of formula (1-a).

The diglycidyl ethers of formulas (1) to (9) are prepared by the reaction of epichlorohydrin and a bisphenol of formulas (1-b) to (9-b):

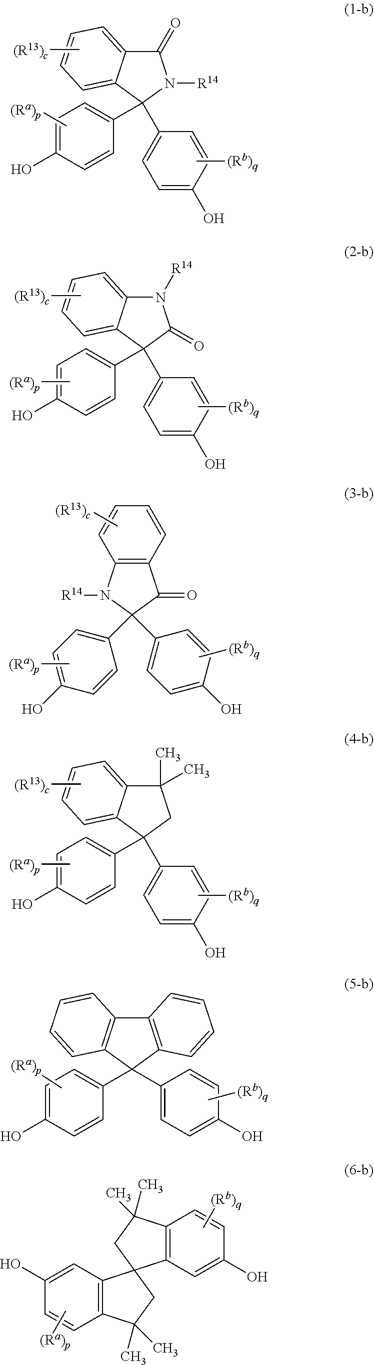

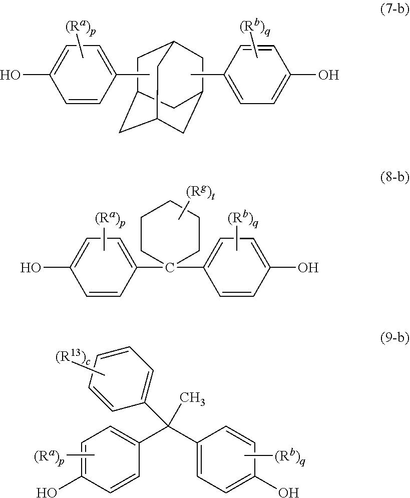

wherein R^{a}, R^{b}, p, q, R¹³, R¹⁴, c, R^{g}, and t are as defined in formulas (1) to (9).

The bisphenols of formula (1-b) to (9-b) can have a purity of 90 to 99.9%, preferably 92 to 99.9%, more preferably 95 to 99.9%, or 97 to 99.9%. In some embodiments, the bisphenols of formula (1-b) to (9-b) can have a purity of 92 to 99.5%, 92 to 99.5%, or 97 to 99.8.

In some embodiments, the diglycidyl ethers of formulas (1-a), (2-a), (3-a), (4-a), (5-a), (6-a), (7-a), (8-a), (8-a′), (8-a″) to (9-a) are prepared by the reaction of epichlorohydrin and a bisphenol of formulas (1-c), (2-c), (3-c), (4-c), (5-c), (6-c), (7-c), (8-c), (8-c′), (8-c″), or (9-c).

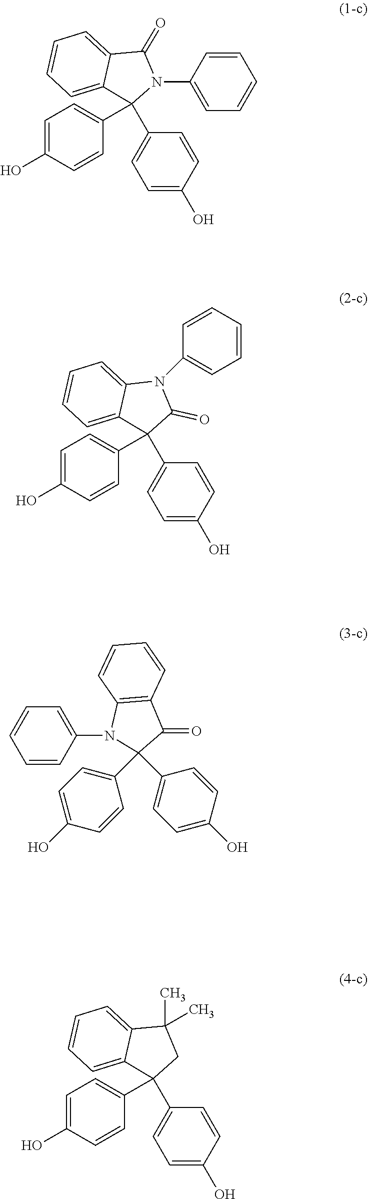 [SMILES: O=C1c2ccccc2C(c2ccc(O)cc2)(c2ccc(O)cc2)N1c1ccccc1 ; O=C1N(c2ccccc2)c2ccccc2C1(c1ccc(O)cc1)c1ccc(O)cc1 ; O=C1c2ccccc2N(c2ccccc2)C1(c1ccc(O)cc1)c1ccc(O)cc1 ; CC1(C)CC(c2ccc(O)cc2)(c2ccc(O)cc2)c2ccccc21]

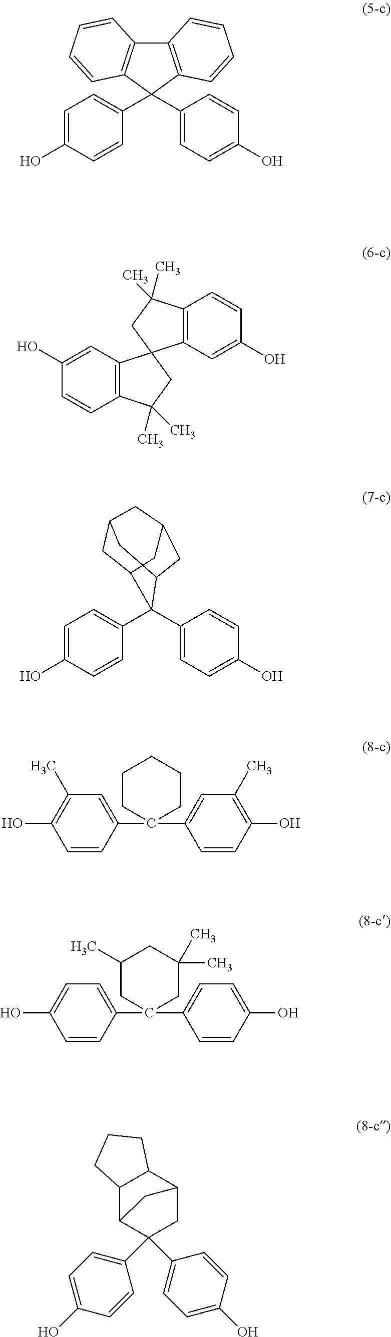 [SMILES: Oc1ccc(C2(c3ccc(O)cc3)c3ccccc3-c3ccccc32)cc1 ; CC1(C)CC2(CC(C)(C)c3ccc(O)cc32)c2cc(O)ccc21 ; Oc1ccc(C2(c3ccc(O)cc3)C3CC4CC(C3)CC2C4)cc1 ; Cc1cc(C2(c3ccc(O)c(C)c3)CCCCC2)ccc1O ; CC1CC(C)(C)CC(c2ccc(O)cc2)(c2ccc(O)cc2)C1 ; Oc1ccc(C2(c3ccc(O)cc3)CC3CC2C2CCCC32)cc1]

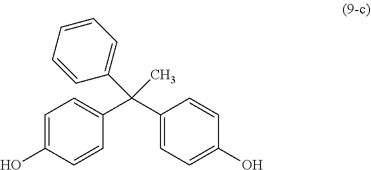 [SMILES: CC(c1ccccc1)(c1ccc(O)cc1)c1ccc(O)cc1]

The bisphenols of formula (1-c), (2-c), (3-c), (4-c), (5-c), (6-c), (7-c), (8-c), (8-c′), (8-c″), or (9-c) can have a purity of 90 to 99.9%, or 92 to 99.9%, or 95 to 99.9%, or 97 to 99.9%. In some embodiments, the bisphenols of formula (1-c), (2-c), (3-c), (4-c), (5-c), (6-c), (7-c), (8-c), (8-c′), (8-c″), or (9-c) can have a purity of 92 to 99.5%, 92 to 99.5%, or 97 to 99.8.

In particular, a bisphenol or combination of bisphenols is reacted with epichlorohydrin and a catalyst for a period of time and at a temperature to allow the reaction to proceed to a first reaction mixture comprising the di-chlorohydrin of formula (1-d) to (9-d):

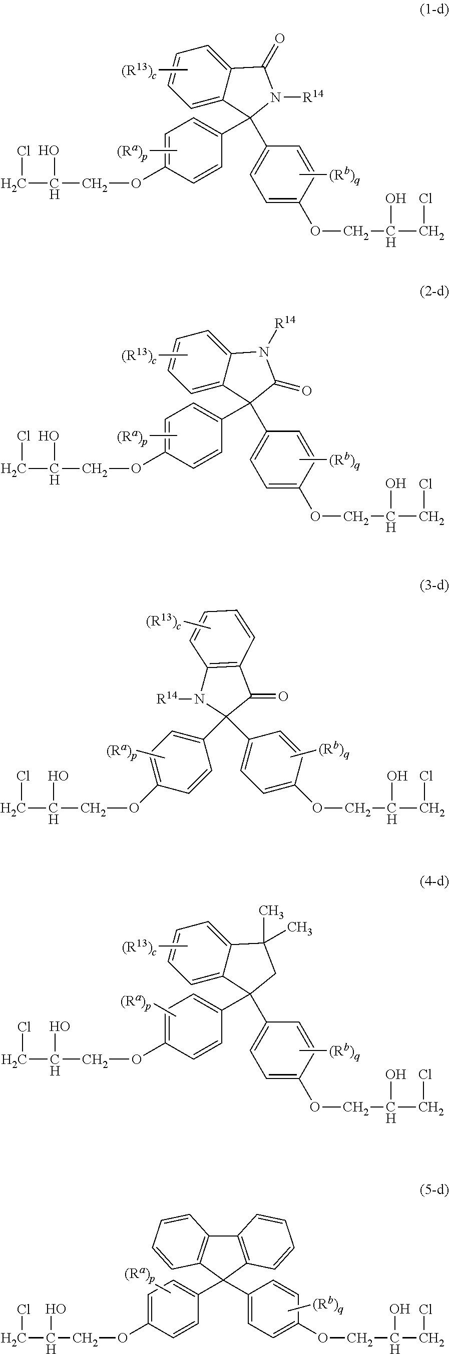

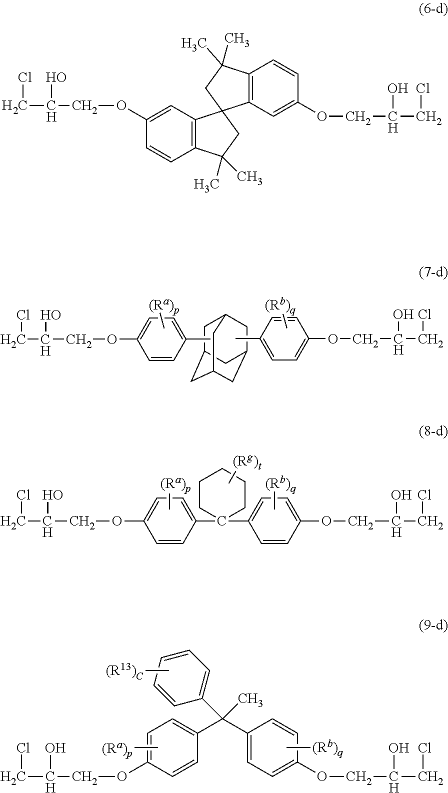

wherein R^{a}, R^{b}, p, q, R¹³, R¹⁴, c, R^{g}, and t are as defined in formulas (1) to (9). In a specific embodiment, the di-chlorohydrins of the diglycidyl ethers of formulas (1-a), (2-a), (3-a), (4-a), (5-a), (6-a), (7-a), (8-a), (8-a′), (8-a″), or (9-a) are formed.

Other compounds can be present in the first reaction mixture, including the diglycidyl ethers of formulas (1) to (9), and the corresponding the mono-substituted chlorohydrins of formula (1-e) to (9-e).

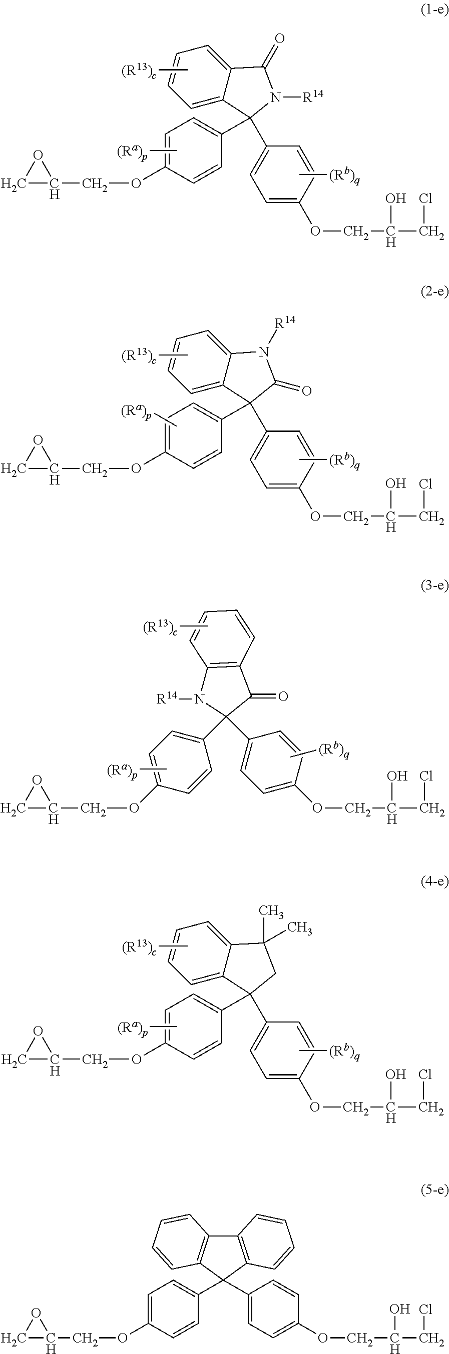

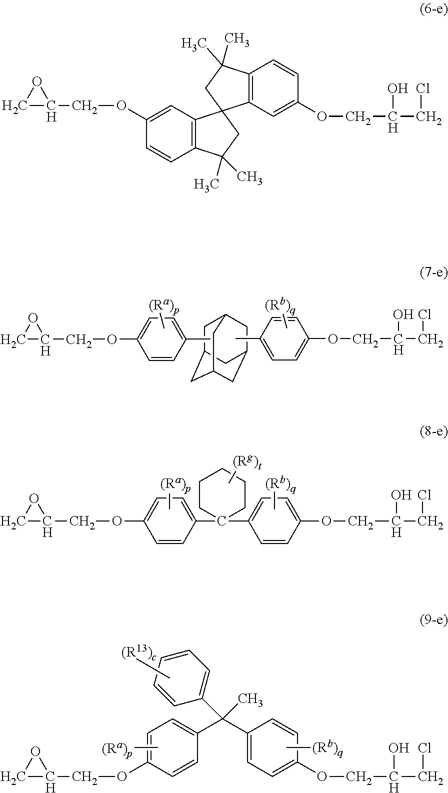

wherein R^{a}, R^{b}, p, q, R¹³, R¹⁴, c, R^{g}, and t are as defined in formulas (1) to (9). In a specific embodiment, the mono-substituted chlorohydrins of the diglycidyl ethers of formulas (1-a), (2-a), (3-a), (4-a), (5-a), (6-a), (7-a), (8-a), (8-a′), (8-a″), or (9-a) are formed. In specific embodiments, the molar ratio of the bisphenol of formula (1-b) to (9-b)/epichlorohydrin can be 1/20 to 1/80, or 1/30 to 1/70, or 1/35 to 1/65.

The reaction can occur in a suitable apparatus. The reaction can occur with agitating. The agitating can be with any suitable apparatus, such as in a stirred vessel. The reaction of epichlorohydrin and the bisphenol is in the presence of a catalyst that can act as a phase transfer agent. A wide variety of catalysts can be used, for example quaternary ammonium salts, quaternary phosphonium salts, sulphoxides, sulpholanes, guanidinium salts, and imidazolium salts. Among the quaternary ammonium and quaternary phosphonium salts that can be used are those of formula (10), among the guanidinium salts that can be used are those of formula (11) and among the imidazolium salts are those of formula (12)

(R¹)(R²)(R³)(R⁴)₄Q⁺X (10)

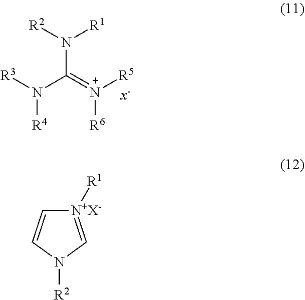

wherein in formulas (10), (11), and (12), each R¹, R², R³, R⁴, R⁵, and R⁶ is the same or different, and is a C₁₋₁₈ alkyl, C₆₋₁₂ aryl, C₇₋₁₂ arylalkylene, or C₇₋₁₂ alkylarylene; Q is a nitrogen or phosphorus atom; and X is an anion, such as a halogen atom, hydroxide, acetate, citrate, or the like.

Exemplary catalysts of the foregoing types include tetramethyl ammonium chloride, tetramethyl ammonium bromide, trimethylbenzyl ammonium chloride, triethylbenzyl ammonium chloride, cetyltrimethyl ammonium bromide, methyltrioctyl ammonium chloride, trioctylbenzyl ammonium chloride, tributylbenzyl ammonium chloride, tetrabutyl ammonium bromide, tetraphenylphosphonium halides, methyltriphenylphosphonium iodide, and ethyltriphenyl phosphonium chloride, dimethylsulfoxide, diphenylsulfoxide, dibutylsulfoxide, sulfolane, 2,4-dimethylsulfolane, dimethylsulfone, diphenylsulfone, hexaethyl guanidinium chloride, and 1-butyl-3-methylimidazolium chloride. Specific catalysts are hexaethylguanidium chloride and tetrabutylammonium bromide.

The catalyst can be present in an amount from 0.5 to 80 mol %, based on the based on the moles of the bisphenol of formula (1-b) to (9-b) or (1-c) to (9-c). The concentration of the catalyst can be adjusted depending on the catalyst, and the optimum concentration can vary from catalyst to catalyst, as can be readily determined by one of ordinary skill in the art without undue experimentation. In an example, the catalyst is hexaethylguanidium chloride and the catalyst is present at 2 to 30 mole percent, based on the moles of the bisphenol of formula (1-c) to (9-c). In another example, the catalyst is tetrabutylammonium bromide and the catalyst is present at 10 to 60 mole percent, based on the bisphenol of formula (1-c) to (9-c).

The reaction time and temperature can vary depending on the bisphenol and catalyst. In embodiments, the reacting the bisphenol and the epichlorohydrin in the presence of the catalyst is for 2 to 9 hours at 30 to 80° C., or 2.5 to 7 hours at 50 to 75° C. The unreacted epichlorohydrin can be removed from the first reaction mixture using any suitable method, such as distilling.

After the formation of the first reaction mixture containing the bis(chlorohydrin) (and other components, for example the mono-substituted chlorohydrins and the diglycidyl ethers), a base is added to the first reaction mixture to provide a second reaction mixture. Suitable bases include, but are not limited to, carbonates (e.g., sodium bicarbonate, ammonium carbonate, or dissolved carbon dioxide), and hydroxide bases (e.g., sodium hydroxide, potassium hydroxide, or ammonium hydroxide). The base can be added as a powder (e.g., powdered sodium hydroxide). The base can be sodium hydroxide or potassium hydroxide, for example. The base can be added over a period of 15 minutes to 3 hours, or over 30 minutes to 3 hours to provide a second reaction mixture. The second reaction mixture can be agitated for a selected time period (e.g., 2 to 5 hours or 3 to 4 hours) at a selected temperature at 40 to 60° C., or at 45 to 55° C. to provide the as-synthesized diglycidyl ethers of formulas (1) to (9) or (la) to (9a).

The as-synthesized diglycidyl ether of formula (1) to (9) or (1-a), (2-a), (3-a), (4-a), (5-a), (6-a), (7-a), (8-a), (8-a′), (8-a″) to (9-a) can have a purity of 97% or greater, as determined by high performance liquid chromatography (HPLC). An HPLC method is described in the Examples below.

The as-synthesized diglycidyl ethers of formulas (1) to (9) or (1-a), (2-a), (3-a), (4-a), (5-a), (6-a), (7-a), (8-a), (8-a′), (8-a″) to (9-a) can have a hydrolyzable chloride content of less than 2,000, less than 1,000, less than 800, or between 300 to 7000 parts per million (ppm).

The as-synthesized diglycidyl ethers of formulas (1) to (9) or (1-a), (2-a), (3-a), (4-a), (5-a), (6-a), (7-a), (8-a), (8-a′), (8-a″) to (9-a) can have an epoxy equivalent weight (EEW) of 202 to 265 grams per equivalent (g/eq). EEW is the weight of the diglycidyl ethers in grams that contains one equivalent of epoxy groups.

The as-synthesized diglycidyl ethers of formulas (1) to (9) or (1-a), (2-a), (3-a), (4-a), (5-a), (6-a), (7-a), (8-a), (8-a′), (8-a″) to (9-a) can include less than 1% of corresponding dimer products, as measured by high performance liquid chromatography.

The as-synthesized diglycidyl ether of formula (1) to (9) or (1-a), (2-a), (3-a), (4-a), (5-a), (6-a), (7-a), (8-a), (8-a′), (8-a″) to (9-a) can include less than 1 total wt % of a di-chlorohydrin of formula (1-d) to (9-d) and the mono-substituted chlorohydrin of formula (1-d) to (9-d). Similarly, the as-synthesized diglycidyl ether of formula (1) to (9) or (1-a), (2-a), (3-a), (4-a), (5-a), (6-a), (7-a), (8-a), (8-a′), (8-a″) to (9-a) can include less than 1 total wt % of the corresponding di-chlorohydrin or the corresponding mono-substituted chlorohydrin, or both.

The as-synthesized diglycidyl ether of formula (1) to (9) or (1-a), (2-a), (3-a), (4-a), (5-a), (6-a), (7-a), (8-a), (8-a′), (8-a″) to (9-a) can have a residual moisture content of 0.01 to 0.5 wt %, or 0.1 to 0.4 wt %.

The as-synthesized diglycidyl ether of formula (1) to (9) or (1-a), (2-a), (3-a), (4-a), (5-a), (6-a), (7-a), (8-a), (8-a′), (8-a″) to (9-a) made by the processes described herein can be substantially free of oligomeric impurities. Accordingly, the as-synthesized diglycidyl ether can have an oligomer impurity content of 3% or less, 2% or less, 1% or less, 0.5% or less, 0.4% or less, 0.3% or less, 0.2% or less, or 0.1% or less, as determined by high performance liquid chromatography.

In an embodiment, the as-synthesized diglycidyl ether of formula (1) to (9) or (1-a), (2-a), (3-a), (4-a), (5-a), (6-a), (7-a), (8-a), (8-a′), (8-a″) to (9-a) have at least two, or at least three of the above-described properties, for example all three of the recited purity, di- and mono-chlorohydrin content, and hydrolyzable chloride content.

The as-synthesized diglycidyl ether can be isolated from the reaction mixture, by methods such as distillation to remove solvents, centrifugation, or filtration, to provide an isolated diglycidyl ether.

Further disclosed herein are diglycidyl ethers of formulas (1) to (9) or (1-a), (2-a), (3-a), (4-a), (5-a), (6-a), (7-a), (8-a), (8-a′), (8-a″) to (9-a) having one or more of the properties described below. Also disclosed are isolated diglycidyl ethers of formulas (1) to (9) or (1-a), (2-a), (3-a), (4-a), (5-a), (6-a), (7-a), (8-a), (8-a′), (8-a″) to (9-a) having one or more of the properties described below.

The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can have a purity of 97% or greater, preferably a purity of 98% of greater, as determined by high performance liquid chromatography.

The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can have less than 1 total wt % of a di-chlorohydrin of formula (1-d) to (9-d) and a mono-substituted chlorohydrin of formula (1-e) to (9-e), as determined by HPLC.

The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can have a hydrolyzable chloride content of less than 2,000, less than 1,000, or less than 800 ppm

The diglycidyl ethers or isolated diglycidyl ethers of formula (1-a), (2-a), and (3-a), for example, can have an EEW of 263 g/eq, 262 g/eq, 261 g/eq, 260 g/eq, 259 g/eq, 258 g/eq, 257 g/eq, 256 g/eq, 255 g/eq, 254 g/eq, 253 g/eq, or 252.7 g/eq. The diglycidyl ethers of formulas (1-a), (2-a), and (3-a), for example, can have an epoxy equivalent weight of 252.6 g/eq.

The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can have an epoxy equivalent weight corresponding to a purity of the diglycidyl ether of 96% purity or greater, 97% purity or greater, 98% purity or greater, 99% purity or greater, 99.5% purity or greater, or 100% purity.

The diglycidyl ethers or isolated diglycidyl ether of formula (1) to (9) made by the processes described herein can contain less than 1 total wt % of a di-chlorohydrin of formula (1-d) to (9-d) and a mono-substituted chlorohydrin of formula (1-e) to (9-e). The diglycidyl ethers or isolated diglycidyl ether of formula (1-a), (2-a), (3-a), (4-a), (5-a), (6-a), (7-a), (8-a), (8-a′), (8-a″) to (9-a) can contain less than 1 total wt % of the corresponding di-chlorohydrin of formula (1-d) to (9-d) and the corresponding mono-substituted chlorohydrin of formula (1-e) to (9-e).

The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can be substantially free of oligomeric impurities. The diglycidyl ether can have an oligomer impurity content of 3% or less, 2% or less, 1% or less, 0.5% or less, 0.4% or less, 0.3% or less, 0.2% or less, or 0.1% or less, as determined by high performance liquid chromatography. The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can have less than 1 wt % of the corresponding dimer, as measured by high performance liquid chromatography. The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can have less than 50, or less than 40, or less than 20, or less than 10, or less than 5 ppm of residual epichlorohydrin. The diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) can have a residual moisture content of 0.01 to 0.5 wt %, or 0.1 to 0.4 wt %.

In an embodiment, the diglycidyl ethers or isolated diglycidyl ethers made by the processes described herein of formulas (1) to (9) or (1-a), (2-a), (3-a), (4-a), (5-a), (6-a), (7-a), (8-a), (8-a′), (8-a″) to (9-a) have at least two, or at least three of the above-described properties, for example all three of the recited purity, di- and mono-chlorohydrin content, and hydrolyzable chloride content.

The diglycidyl ethers of formulas (1) to (9) or (1-a), (2-a), (3-a), (4-a), (5-a), (6-a), (7-a), (8-a), (8-a′), (8-a″) to (9-a) (which encompasses diglycidyl ethers having the properties described herein, preferably the as-synthesized or isolated diglycidyl ethers made by the methods described herein) are useful in curable compositions for the manufacture of a wide variety of articles. Thus, the diglycidyl ethers can be combined, e.g., blended, with one or more additional components to provide curable compositions. For example, the curable compositions can further include a curing promoter, auxiliary epoxy resin, filler, reinforcing fiber, rubber, solvent, or a combination comprising at least one of the foregoing. In an embodiment, the curable compositions can include a diglycidyl ether of formula (1) to (9) or (1-a), (2-a), (3-a), (4-a), (5-a), (6-a), (7-a), (8-a), (8-a′), (8-a″) to (9-a) in an amount of 1 wt % to 99.9 wt %, 3 wt % to 50 wt %, 5 wt % to 40 wt %, or 10 wt % to 30 wt %, based on total weight of the curable composition and these additional components.

The curable composition can include a curing promoter. The term “curing promoter” as used herein encompasses compounds having a role in curing epoxy resins that are variously described as those of a hardener, a hardening accelerator, a curing catalyst, and a curing co-catalyst, among others. The compounds known as hardeners react with the epoxy groups and/or the secondary hydroxyl groups of the epoxy polymer. Suitable hardeners are known in the art and include, for example, amines, dicyandiamide, polyamides, amidoamines, Mannich bases, anhydrides, phenol-formaldehyde resins, carboxylic acid functional polyesters, polysulfides, polymercaptans, isocyanates, cyanate esters, and the like. A combination comprising at least one of the foregoing hardeners can be used. For example, the curable composition can comprise 1 to 99.9 wt % of the diglycidyl ether, and 0.1 to 50 wt % of the curing promoter, based on the total weight of the curable composition.

A wide variety of auxiliary epoxy resins can used, based on compatibility with the diglycidyl ethers of formulas (1) to (9) or (1-a), (2-a), (3-a), (4-a), (5-a), (6-a), (7-a), (8-a), (8-a′), (8-a″) to (9-a), and the desired properties of the cured composition. Some specific auxiliary epoxy resins include a 3,3′,5,5′-tetramethyl-[1,1′-biphenyl]-4,4′-glycidyl ether (biphenyl type epoxy) cresol novolak type epoxy; a phenol novolak epoxy; a flame-retardant bisphenol epoxy a brominated bisphenol A epoxy; a tetrabromobisphenol A epoxy; a brominated polyfunctional bisphenol A epoxy; a brominated novolac bisphenol A epoxy resin; a bisphenol A diglycidyl ether; an epoxy resin having a DCPD (dicylopentadiene) skeleton; an epoxy resin having a naphthalene skeleton, including 1,6-bis(2,3-epoxy propoxy)naphthalene; a triphenylmethane type epoxy; multi-functionalized epoxies; tetraglycidyl ether diaminodiphenylmethane; RE-310 and RE-303 from Nippon Kayaku Co.; Epiclon 153 from Dainippon Ink & Chemicals, Inc.; VG3101 from Mitsui Petrochemical Industries, Ltd., EEW=209.

The curable composition can include inorganic fillers. Suitable inorganic fillers are known and can include, for example, alumina, silica (including fused silica and crystalline silica), boron nitride (including spherical boron nitride), aluminum nitride, silicon nitride, magnesia, magnesium silicate, glass fibers, glass mat, or a combination comprising at least one of the foregoing. Suitable glass fibers include those based on E, A, C, ECR, R, S, D, and NE glasses, as well as quartz. The glass fiber can have a diameter of 2 to 30 micrometers, or 5 to 25 micrometers, or 5 to 15 micrometers. The length of the glass fibers before compounding can be 2 to 7 millimeters, or 1.5 to 5 millimeters. Alternatively, longer glass fibers or continuous glass fibers can be used. Adhesion promoters can be used and include chromium complexes, silanes, titanates, zircon-aluminates, propylene maleic anhydride copolymers, reactive cellulose esters and the like. Suitable glass fiber is commercially available from suppliers including, for example, Owens Corning, Nippon Electric Glass, PPG, and Johns Manville.

The curable composition can include impact modifiers, for example elastomers or rubbers. Exemplary rubbers include carboxyl-terminated butadiene acrylonitrile liquid polymers (CTBN), acrylonitrile-butadiene rubber (NBR), phenol-terminated butadiene-acrylonitrile liquid polymers (PTBN), secondary amine-terminated butadiene-acrylonitrile liquid polymers (ATBN), hydroxyl-terminated butadiene-acrylonitrile liquid polymers (HTBN), carboxyl-terminated butadiene liquid polymers (CTB), and various block copolymers, including SBS rubbers (styrene-butadiene-styrene block copolymers), SEP rubbers (styrene-ethylene/propylene block copolymers), SEBS rubbers (styrene ethylene/butylene-styrene block copolymers), and liquid polyolefin hydrocarbons. Other rubbers that can be used included a butadiene-acrylonitrile copolymerization rubber polyvinyl-acetal polymer and SBS.

The curable composition can include solvents. Suitable solvents can include, for example, a C₃₋₈ ketone, an N,N-di(C₂₋₄alkyl)amide, a cyclic or acyclic di(C₂₋₄alkyl) ether that can optionally further include one or more ether oxygen atoms within the alkyl groups and one or more hydroxy group substituents on the alkyl groups, a C₆₋₁₂ aromatic hydrocarbon, preferably unhalogenated (i.e., does not include any fluorine, chlorine, bromine, or iodine atoms), a C₁₋₃ chlorinated hydrocarbon, a (C₁₋₄ alkyl) (C₁₋₄alk)anoate, a (C₅₋₆ alkyl)cyanide, or a combination comprising at least one of the foregoing. Specific solvents include, for example, acetone, methyl ethyl ketone, methyl isobutyl ketone, dimethylformamide, dimethylacetamide, N-methyl-2-pyrrolidone (Chemical Abstracts Service Registry No. 872-50-4), tetrahydrofuran, ethylene glycol monomethylether, dioxane (preferably tetrahydrofuran and dioxane), benzene, toluene, xylenes, methyl acetate, ethyl acetate, methyl propionate, ethyl propionate, acetonitrile, propionitrile, and butyronitrile. A combination comprising any of the foregoing can be used. Specific solvents that can be used are acetone, methyl ethyl ketone, methyl isobutyl ketone, toluene, and N-methyl-2-pyrrolidone, ethylene glycol monomethyl ether, and dimethylformamide.

When a solvent is used, the curable composition can comprise 2 to 100 parts by weight of the solvent, based on 100 parts by weight total of the diglycidyl ether and curing promoter. Specifically, the solvent amount can be 5 to 80, or 10 to 60, or 20 to 40 parts by weight, based on 100 parts by weight total of the diglycidyl ether, any optional auxiliary epoxy polymers, and any curing promoter. The solvent can be chosen, in part, to adjust the viscosity of the curable composition. Thus, the solvent amount can depend on variables including the type and amount of the diglycidyl ether, the type and amount of any auxiliary epoxy polymers, the type and amount of curing promoter, and the processing temperature used for forming an article, in particular impregnation of a reinforcing structure with the curable composition.

The curable compositions can further include flame retardants, dyes, pigments, colorants, antioxidants, heat stabilizers, light stabilizers, plasticizers, lubricants, flow modifiers, drip retardants, antiblocking agents, antistatic agents, flow-promoting agents, processing aids, substrate adhesion agents, mold release agents, toughening agents, low-profile additives, stress-relief additives, or combinations comprising at least one of the foregoing. The foregoing additives can be present individually in an amount of 0.001 to 8 wt %, or 0.005 to 5 wt %, although the total amount of the additives generally is not greater than 10 wt. %, based on the total weight of the diglycidyl ether and the additives. Exemplary additional additives include, for example, poly(tetrafluoroethylene) (PTFE); natural carnauba; 6,6′-(sulfonyl)bis(3,4-dihydro-3-phenyl-2H-1,3-benzoxazine); and polyhedral oligomeric silsesquioxane (POSS) based components; and the like.

The curable compositions can be subjected to various treatments to cure the composition (e.g., initiate reaction of the diglycidyl ether with a curing promoter, such as a polyamine). There is no particular limitation on the method by which the composition can be cured. The composition can, for example, be cured thermally or by using irradiation techniques, including ultraviolet (UV) irradiation and electron beam irradiation. When heat curing is used, the temperature selected can be 80 to 300° C., and preferably 120 to 240° C. The heating period can be 1 minute to 10 hours, though such heating period can advantageously be 1 minute to 6 hours, more preferably 3 hours to 5 hours. Such curing can be staged to produce a partially cured and often tack-free polymer, which then is fully cured by heating for longer periods or temperatures within the aforementioned ranges.

Also provided is a cured composition comprising the product obtained by curing the curable composition. The cured compositions can have a Tg of 145° C. to 420° C. Differential scanning calorimetry (DSC) can be conducted with a heating rate of 10° C./minute or 20° C./minute. The cured composition can exhibit a single Tg, as opposed to two or more Tgs, indicating that the diglycidyl ether is covalently bound to an epoxy polymer matrix of the cured composition. In other words, the diglycidyl ether may not exist as a separate phase within the epoxy polymer matrix. Depending on the type and relative amounts of diglycidyl ether components and curing promoters, the glass transition temperature can 100° C. to 400° C., or 150° C. to 300° C., for example.

The cured compositions can exhibit good impact strength. In some embodiments, the cured composition exhibits an unnotched Izod impact strength of at least 400 joules per meter, or 400 to 600 joules per meter, or 450 to 550 joules per meter, or 480 to 520 joules per meter, as measured at 23° C. with a hammer energy of 2 foot-pounds in accordance with ASTM D 4812-06. The cured compositions can exhibit good ductility. The cured compositions can exhibit good fracture toughness, unnotched Izod impact strength, and good tensile elongation. The cured compositions can exhibit increased char formation on pyrolysis. The cured compositions can exhibit low moisture absorption. The cured compositions can exhibit decreased shrinkage upon curing. The cured compositions can exhibit good dielectric properties. For example, the cured compositions can exhibit a dielectric constant of 2.8 to 3.2, or 2.9 to 3.1, or 3.00 to 3.06, as measured at 1,000 megahertz in accordance with IPC-TM-650 2.5.5.9. The cured compositions can exhibit a loss tangent of 0.011 to 0.017, or 0.012 to 0.016, or 0.013 to 0.015, as measured at 1,000 megahertz in accordance with IPC-TM-650 2.5.5.9. The cured compositions can exhibit a water absorption of 5 wt % or less, or 4 wt % or less, or 3 wt % or less, or 2 wt % or less, measured after immersion in deionized water at 80° C. for 250 hours. The cured composition can preferably exhibit a coefficient of thermal expansion (CTE) below its glass transition temperature (Tg) of not greater than 30 micrometer/meter-° C. (μm/m-° C.), preferably not greater than 25 μm/m-° C., more preferably not greater than 20 μm/m-° C. The cured compositions can have a haze of 10% or less, or 8% or less and a transmittance of about 75% or more, or 85% or more. Haze and transmittance can be measured on 5 millimeter color plaques according to ASTM D 1003-00 using a BYK Gardner haze-guard dual with a D65 light source. The cured compositions can exhibit a number of these or additional advantageous properties simultaneously.

Also provided is an article comprising the cured composition. In a specific embodiment, the article can be a coating, encapsulant, adhesive, or a matrix polymer for a composite. The articles are especially useful in an electronic device, such as a chip encapsulant or a dielectric material for a circuit.

However, the cured compositions can be used in a variety of applications and articles, including any applications where conventional epoxides are currently used. Exemplary uses and applications include coatings such as protective coatings, sealants, weather resistant coatings, scratch resistant coatings, and electrical insulative coatings; adhesives; binders; glues; composite materials such as those using carbon fiber and fiberglass reinforcements. When used as a coating, the cured compositions can be disposed on a surface of a variety of underlying substrates. For example, cured compositions can be disposed on a surface of metals, plastics, glass, fiber sizings, ceramics, stone, wood, or a combination comprising at least one of the foregoing. The cured compositions can be used as a coating on a surface of a metal container, such as those commonly used for packaging and containment in the paint and surface covering industries. In some instances, the coated metal is aluminum or steel.

Other articles including the cured compositions include, for example, electrical components, computer components, and automotive, aircraft, and watercraft exterior and interior components. In some embodiments, the cured compositions are useful in composite materials for use in the aerospace industry. Additional applications for the curable compositions include, for example, acid bath containers; neutralization tanks; aircraft components; bridge beams; bridge deckings; electrolytic cells; exhaust stacks; scrubbers; sporting equipment; stair cases; walkways; automobile exterior panels such as hoods and trunk lids; floor pans; air scoops; pipes and ducts, including heater ducts; industrial fans, fan housings, and blowers; industrial mixers; boat hulls and decks; marine terminal fenders; tiles and coatings; building panels; business machine housings; trays, including cable trays; concrete modifiers; dishwasher and refrigerator parts; electrical encapsulants; electrical panels; tanks, including electrorefining tanks, water softener tanks, fuel tanks, and various filament-wound tanks and tank linings; furniture; garage doors; gratings; protective body gear; luggage; outdoor motor vehicles; pressure tanks; printed circuit boards; optical waveguides; radomes; railings; railroad parts such as tank cars; hopper car covers; car doors; truck bed liners; satellite dishes; signs; solar energy panels; telephone switchgear housings; tractor parts; transformer covers; truck parts such as fenders, hoods, bodies, cabs, and beds; insulation for rotating machines including ground insulation, turn insulation, and phase separation insulation; commutators; core insulation and cords and lacing tape; drive shaft couplings; propeller blades; missile components; rocket motor cases; wing sections; sucker rods; fuselage sections; wing skins and fairings; engine narcelles; cargo doors; tennis racquets; golf club shafts; fishing rods; skis and ski poles; bicycle parts; transverse leaf springs; pumps, such as automotive smog pumps; electrical components, embedding, and tooling, such as electrical cable joints; wire windings and densely packed multi-element assemblies; sealing of electromechanical devices; battery cases; resistors; fuses and thermal cut-off devices; coatings for printed wiring boards; casting items such as capacitors, transformers, crankcase heaters; small molded electronic parts including coils, capacitors, resistors, and semiconductors; as a replacement for steel in chemical processing, pulp and paper, power generation, and wastewater treatment; scrubbing towers; pultruded parts for structural applications, including structural members, gratings, and safety rails; swimming pools, swimming pool slides, hot-tubs, and saunas; drive shafts for under the hood applications; dry toner polymers for copying machines; marine tooling and composites; heat shields; submarine hulls; prototype generation; development of experimental models; laminated trim; drilling fixtures; bonding jigs; inspection fixtures; industrial metal forming dies; aircraft stretch block and hammer forms; vacuum molding tools; flooring, including flooring for production and assembly areas, clean rooms, machine shops, control rooms, laboratories, parking garages, freezers, coolers, and outdoor loading docks; electrically conductive compositions for antistatic applications; for decorative flooring; expansion joints for bridges; injectable mortars for patch and repair of cracks in structural concrete; grouting for tile; machinery rails; metal dowels; bolts and posts; repair of oil and fuel storage tanks, and numerous other applications including optical applications such as lenses, light emitting diode (LED) encapsulants, and other optical and non-optical applications.

Processes useful for preparing the articles and materials include those generally known to the art for the processing of thermosetting polymers. Such processes have been described in the literature as in, for example, Engineered Materials Handbook, Volume 1, Composites, ASM International Metals Park, Ohio, copyright 1987 Cyril A. Dostal Senior Ed, pp. 105-168 and 497-533, and “Polyesters and Their Applications” by Bjorksten Research Laboratories, Johan Bjorksten (pres.) Henry Tovey (Ch. Lit. Ass.), Betty Harker (Ad. Ass.), James Henning (Ad. Ass.), Reinhold Publishing Corporation, New York, 1956. Processing techniques include polymer transfer molding; sheet molding; bulk molding; pultrusion; injection molding, including reaction injection molding (RIM); atmospheric pressure molding (APM); casting, including centrifugal and static casting open mold casting; lamination including wet or dry lay up and spray lay up; also included are contact molding, including cylindrical contact molding; compression molding; including vacuum assisted polymer transfer molding and chemically assisted polymer transfer molding; matched tool molding; autoclave curing; thermal curing in air; vacuum bagging; pultrusion; Seeman's Composite Resin Infusion Manufacturing Processing (SCRIMP); open molding, continuous combination of polymer and glass; and filament winding, including cylindrical filament winding. In certain embodiments, an article can be prepared from the disclosed curable compositions via a polymer transfer molding process.

In some embodiments, the cured compositions are composites formed from the curable compositions and a reinforcing filler. Methods of forming composites for use in printed circuit boards are known in the art and are described, for example, in U.S. Pat. No. 5,622,588 to Weber, U.S. Pat. No. 5,582,872 to Prinz, and U.S. Pat. No. 7,655,278 to Braidwood. For example, Methods of forming a composite can include impregnating a reinforcing structure with a curable composition; partially curing the curable composition to form a prepreg; and laminating a plurality of prepregs; wherein the curable composition comprises the diglycidyl ether, a curing promoter, and optionally, one or more additional additives.

Reinforcing structures suitable for prepreg formation are known in the art. Suitable reinforcing structures include reinforcing fabrics. Reinforcing fabrics include those having complex architectures, including two- or three-dimensional braided, knitted, woven, and filament wound. The curable composition is capable of permeating such complex reinforcing structures. The reinforcing structure can comprise fibers of materials known for the reinforcement of plastics material, for example fibers of carbon, glass, metal, and aromatic polyamides. Suitable reinforcing structures are described, for example in “Prepreg Technology,” March 2005, Publication No. FGU 017b (Hexcel Corporation); “Advanced Fibre Reinforced Matrix Products for Direct Processes,” June 2005, Publication No. ITA 272 (Hexcel Corporation); and Bob Griffiths, “Farnborough Airshow Report 2006,” CompositesWorld.com, September 2006. The weight and thickness of the reinforcing structure are chosen according to the intended use of the composite using criteria well known to those skilled in the production of fiber reinforced polymer composites. The reinforced structure can contain various finishes suitable for the epoxy matrix.

The method of forming the composite comprises partially curing the curable composition after the reinforcing structure has been impregnated with it. Partial curing is curing sufficient to reduce or eliminate the wetness and tackiness of the curable composition but not so great as to fully cure the composition. The polymer in a prepreg is customarily in the partially cured state, and those skilled in the thermoset arts, and particularly the reinforced composite arts, understand the concept of partial curing and how to determine conditions to partially cure a polymer without undue experimentation. References herein to properties of the “cured composition” refer to a composition that is substantially fully cured. For example, the polymer in a laminate formed from prepregs is typically substantially fully cured. One skilled in the thermoset arts can determine whether a sample is partially cured or substantially fully cured without undue experimentation. For example, one can analyze a sample by differential scanning calorimetry to look for an exotherm indicative of additional curing occurring during the analysis. A sample that is partially cured will exhibit an exotherm. A sample that is substantially fully cured will exhibit little or no exotherm. Partial curing can be effected by subjecting the curable-composition-impregnated reinforcing structure to a temperature of 133 to 140° C. for 4 to 10 minutes.

Commercial-scale methods of forming composites are known in the art, and the curable compositions described herein are readily adaptable to existing processes and equipment. For example, prepregs are often produced on treaters. The main components of a treater include feeder rollers, a polymer impregnation tank, a treater oven, and receiver rollers. The reinforcing structure (E-glass, for example) is usually rolled into a large spool. The spool is then put on the feeder rollers that turn and slowly roll out the reinforcing structure. The reinforcing structure then moves through the polymer impregnation tank, which contains the curable composition. The varnish impregnates the reinforcing structure. After emerging from the tank, the coated reinforcing structure moves upward through the vertical treater oven, which is typically at a temperature of 175 to 200° C., and the solvent of the varnish is boiled away. The polymer begins to polymerize at this time. When the composite comes out of the tower it is sufficiently cured so that the web is not wet or tacky. The cure process, however, is stopped short of completion so that additional curing can occur when laminate is made. The web then rolls the prepreg onto a receiver roll.

While the above-described curing methods rely on thermal curing, it is also possible to effect curing with radiation, including ultraviolet light and electron beams. Combinations of thermal curing and radiation curing can also be used.

In some embodiments, a composite is formed by a method comprising impregnating a reinforcing structure with a curable composition; partially curing the curable composition to form a prepreg; and laminating a plurality of prepregs; wherein the curable composition comprises the diglycidyl ether, a curing promoter, and optionally, one or more additional additives.

In some embodiments, a printed circuit board comprises a composite formed by impregnating a reinforcing structure with a curable composition; partially curing the curable composition to form a prepreg; and laminating a plurality of prepregs; wherein the curable composition comprises the diglycidyl ether, a curing promoter, and optionally, one or more additional additives.

The compositions and methods described herein are further illustrated by the following non-limiting examples.

### EXAMPLES

The materials listed in Table 1 were used.

TABLE 1

| Component | Description | Source | purity |
|---|---|---|---|
| PPPBP | 3,3′-bis(hydroxyl | SABIC | ≥99% |
| | phenyl)-N-phenyl phthalimidine | | |
| | with an epoxy equivalent weight | | |
| | 252.5 g/eq, CAS Registry No. | | |
| | 22749-87-7 | | |
| ECH | Epichlorohydrin | Fluka | ≥99.5% |
| KOH | Potassium hydroxide | | |
| Na₂SO₃ | Sodium sulfite | | |
| NaOH | Sodium hydroxide | SD Fine | 97% |
| | | Chemical, | |
| | | India | |
| DCM | Dichloromethane | Sigma | 99.5% |
| MeOH | Methanol | MERCK | 99.7% |
| TBAB | Tetrabutylammonium bromide. | Aldrich | ≥98% |
| | Phase transfer catalyst | | |
| BTMACl | Benzyl trimethyl ammonium | | |
| | chloride | | |
| BMIMCl | 1-butyl-3-methylimidazolium | | |
| | chloride | | |
| HEGCl | Hexaethylguanidium chloride | | |

HPLC. The purity of reaction samples and products was evaluated by determining the area percentage of bisphenol glycidyl ether, dimer, oligomer, dichlorohydrin, monochlorohydrin, other related compounds by HPLC. Each peak in the chromatogram was integrated and the purities are reported as the area % purity. Each analysis was repeated with triplicate sample preparation and triplicate injections. The HPLC method used is outlined in Table 2.

TABLE 2
HPLC method

| Method name | PPPBP epoxy | | |
|---|---|---|---|
| Wavelength | 230 nm | | |
| Column | Agilent Zorbax-C18, 4.6 × 150 mm, 5 μm | | |
| Column oven | 30° C. | | |
| temperature | | | |
| Injection Vol. | 5 μL | | |
| Flow rate | 1.00 mL/min | | |
| Data acquisition | 30 mins | | |
| | | % Milli Q water(With | |
| | Time | 0.02% OPA) | % Acetonitrile |
| Gradient Program | 0.00 | 80 | 20 |
| | 10.00 | 80 | 20 |
| | 15.00 | 10 | 90 |
| | 22.00 | 10 | 90 |
| | 23.00 | 80 | 20 |
| | 30.00 | 80 | 20 |

EEW estimation. ¹HNMR spectroscopy was carried out using an Agilent 600 MHz spectrometer and CDCl₃ as solvent.

Hydrolysable Chloride Content. D 1726-03 (standard test method for Hydrolysable Chloride Content of Liquid Epoxy resins) was used for sample preparation. Quantification was done using ion chromatography was used to estimate chlorides, instead of titration.

Residual Epichlorohydrin. Residual epichlorohydrin in the final product was estimated using head space gas chromatography. The quantification of residual ECH in diglycidyl ether of PPPBP was carried out by dissolving the sample in dimethyl sulphoxide and subsequently analyzed using headspace gas chromatography. The method has detection capability up to 5 ppm in sample matrix.

Residual Moisture. Residual moisture was estimated using Karl-Fischer Moisture analysis method (ASTM E203).

The examples below are based on a two-step process for the synthesis of the diglycidyl ether of PPPBP is shown in Scheme 1.

### Step 1. Chlorohydrin Formation

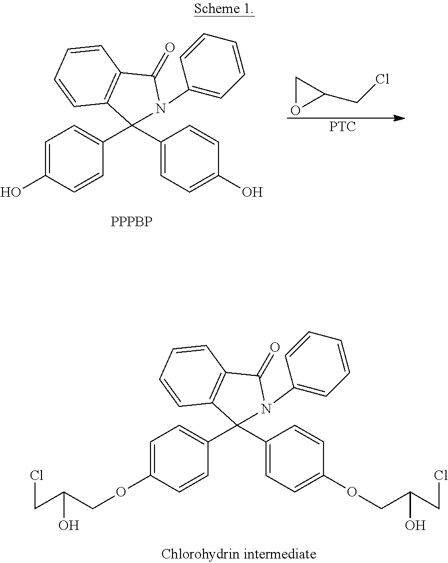

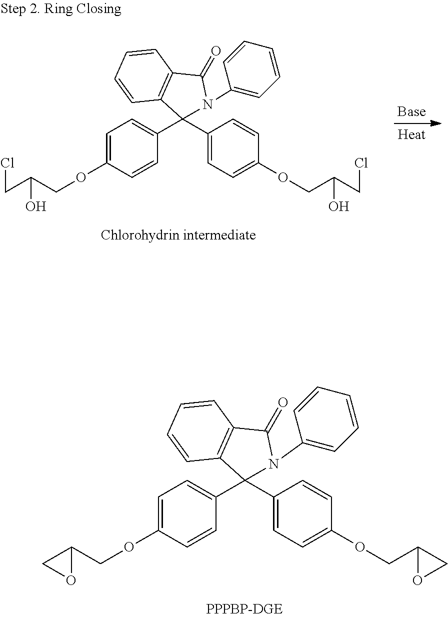 [SMILES: O=C1c2ccccc2C(c2ccc(OCC(O)CCl)cc2)(c2ccc(OCC(O)CCl)cc2)N1c1ccccc1 ; O=C1c2ccccc2C(c2ccc(OCC3CO3)cc2)(c2ccc(OCC3CO3)cc2)N1c1ccccc1]

In particular, in the first step, the reaction proceeds via reaction of PPPBP with epichlorohydrin (ECH) to provide the chlorohydrin intermediate in the presence of a phase transfer catalyst. A mono-substituted chlorohydrin intermediate and final product is also formed in step 1. In a second step, the intermediate is ring-closed in the presence of a base to provide the corresponding diglycidyl ether (DGE) of formula 9-a.

### Example 1. Process A—Comparative

Processes for making a high purity diglycidyl ether of PPPBP are described in PCT/US 15/45004, for example. An exemplary process proceeds as follows. PPPBP is dissolved with a 15 to 25 molar excess of epichlorohydrin (ECH) with respect to PPPBP in the presence of TBAB (tetrabutylammonium bromide, 30 to 60 mol % with respect to PPPBP) by stirring at 40 to 70° C. for 15 to 70 minutes. This step is followed by the slow addition of solid or 50% aqueous NaOH or KOH over a period of 1 to 3.5 hours, followed by a further reaction time of 1 to 4 hours. The resultant reaction mixture is dissolved in dichloromethane (DCM) and washed with water. The aqueous layer is discarded and the DCM/ECH layer is retained after the dissolved alkali is completely removed. The organic layer is subjected to rotary distillation at 50° C. The as-synthesized product thus obtained is a white solid with about 95% yield. This product is characterized by ¹HNMR and HPLC. The purity of the product estimated by HPLC is around 93 to 95% (area %), and contains oligomers and unreacted chlorohydrin as impurities. The as-synthesized product is purified by column chromatography on a silica gel column and using DCM/MeOH (99/1%) mixture as eluent. The final product after isolation has about 99% purity. The purification step results in 50% yield loss.

In view of the final chromatography step being the most labor intensive, time consuming, and costly step, various alternatives were explored to eliminate this step while at the same time obtaining an as-synthesized diglycidyl product with improved purity. Various reaction parameters were examined for both steps of the reaction process, including catalyst type, catalyst loading, ratio of PPPBP to epichlorohydrin, and the mode of addition of the reactants/reagents, for example.

### Example 2. Process B—Improved Formation of Chlorohydrin

In Example 1, the conversion of PPPBP into the chlorohydrin intermediate is not completed before addition of base. It was found that the unreacted PPPBP in the presence of base during the ring closure step results in the formation of oligomers and thereby results in an as-synthesized product of low purity that requires a column purification to obtain the desired product purity. In this example, the parameters used in the first step were adjusted to provide more complete conversion of the PPPBP to the chlorohydrin intermediately before the ring closure step. Accordingly, PPPBP was reacted with 20 to 100 mol % excess of epichlorohydrin in presence of a phase transfer catalyst (TBAB, 50 mol % with respect to PPPBP) at 50° C. for 6 to 8 hrs. The reaction was monitored by analyzing aliquots taken out during the progress of the reaction. The samples were analyzed by HPLC and it was determined the phenolic OH groups were completely converted to the corresponding chlorohydrin intermediate. This step was followed by addition of 50% NaOH solution over a period of 0.5 to 1 hr, followed by further reaction time of 3 to 4 hours. The resultant reaction mixture containing the as-synthesized diglycidyl ether of PPPBP was subsequently treated as in Example 1 to isolate the diglycidyl ether.

### Example 3. Process C—Scale-Up

While the process of Example 2 worked well on laboratory scale, a drop in purity of the final isolated product was observed during epichlorohydrin removal step by vacuum distillation. The process was accordingly further modified.

The conversion of PPPBP to chlorohydrin was performed as described in Process B (Example 2). The reaction was carried out in 1 gram scale and temperature was maintained at 50° C. with the time of reaction same as provided for Process B. Next, unreacted epichlorohydrin was distilled off prior to addition of base. The distillation was carried out at 50° C. and under vacuum (reducing the pressure gradually from 900 milliBar to 1 milliBar). The viscous mass obtained at the end of the process was dissolved in DCM and treated with 50% NaOH solution (2 to 3 moles with respect to PPPBP). The base solution was added dropwise over 30 to 60 minutes and the reaction was allowed to continue at 50° C. The reaction time was adjusted based on the progress of the reaction tracked by periodic HPLC analysis. At the end of the reaction, the as-synthesized product in the DCM solution was washed with water repeatedly until all of the base was removed. The neutral DCM solution was refluxed with aqueous 10% sodium sulfite (20 volumes) at 100° C. using a Dean-Stark apparatus. DCM was collected and the process was allowed to continue for 1 hour. White solid precipitated out in the solution. The precipitate was filtered and the residue was dried in thermostat oven at 100° C. to provide an isolated product. The isolated product was found to have a hydrolysable chloride content of 580 ppm, a residual epichlorohydrin content of less than 5 ppm, and a residual moisture content of 0.25 to 0.35 wt %.

### Example 4. Process D—Solvent

The epoxidation reaction was also carried out in presence of different solvents to perform the reaction at PPPBP/ECH mole ratios with a relatively low amount of ECH. The PPPBP reaction with ECH was carried out at 50° C. using TBAB as catalyst using isopropanol, dichloromethane, dioxane, and DMSO as solvent. The percentage solid was maintained at 30%. The progress of the reaction was tracked by HPLC in a similar manner as discussed above. In these examples, the PPPBP/ECH ratio was maintained at 1/10 with TBAB at 10 mol %.

### Example 5. Process E—Dropwise PPPBP Addition

In Processes A through D, the reactants PPPBP, ECH, and catalyst were added in the first step simultaneously. A variation to these processes was made by adding dropwise PPPBP solution to a reaction flask containing ECH and HEGC1, maintained at 50° C. This way addition of PPPBP could keep the ECH always in an excess amount compared to PPPBP and keep the dimerization at lower levels of ECH. In this process, PPPBP solution was prepared in aqueous NaOH solution. The PPPBP/ECH ratio was kept at 1/60 and HEGCl at 10 mol %. The addition was completed over 2 hours and the reaction was monitored using HPLC.

### Example 6. Phase Transfer Catalyst—Process A

The effect of phase transfer catalyst on the purity of the as-synthesized product was studied, using Process A. The catalysts chosen were tetrabutylammonium bromide (TBAB), benzyltrimethyl ammonium chloride (BTMAC1), 1-butyl-3-methylimidazolium chloride (BMIMC1), and hexaethylguanidium chloride (HEGC1). Each of the four different catalysts was added to a reaction mixture containing PPPBP and ECH at a mole ratio of PPPBP/ECH of 1/20. The catalyst level was maintained at 50 mole %, based on the moles of PPPBP. The reaction was carried out at 50° C. and aliquots were taken out regularly and analyzed by HPLC as described below.

The results of the experiments for Process A are shown in Table 3.

TABLE 3

| | Experiment No. | | |
|---|---|---|---|
| | 4a | 4b | 4c |
| Catalyst Type | BMIMCl | BTMAC | TBAB |
| Purity at the | 93.9 | 96.4 | 94.8 |
| end of reaction | | | |
| (HPLC area %) | | | |

### Example 7. Phase Transfer Catalyst—Process B

Dimers are believed to be formed by the reaction of the epoxy group of the end products with free phenolic OH group of the PPPBP or the intermediates PCH and PE shown below.

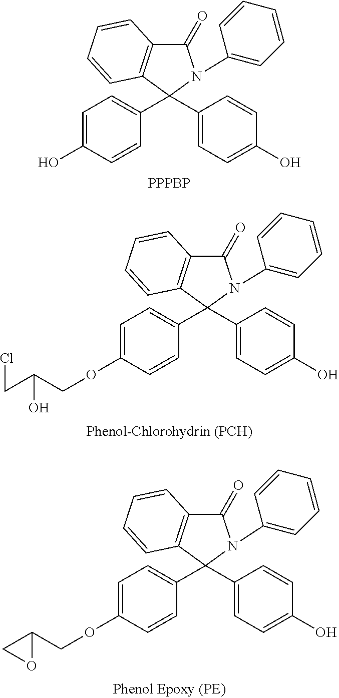

The peaks corresponding to these structures were identified using liquid chromatography-mass spectrometry (LC-MS) and their disappearance with time was tracked using HPLC.

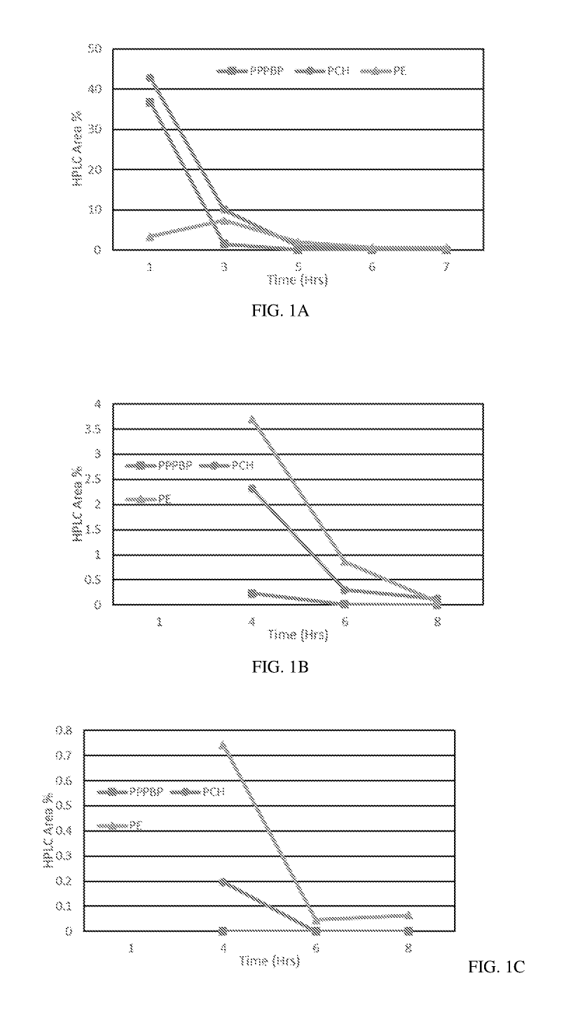

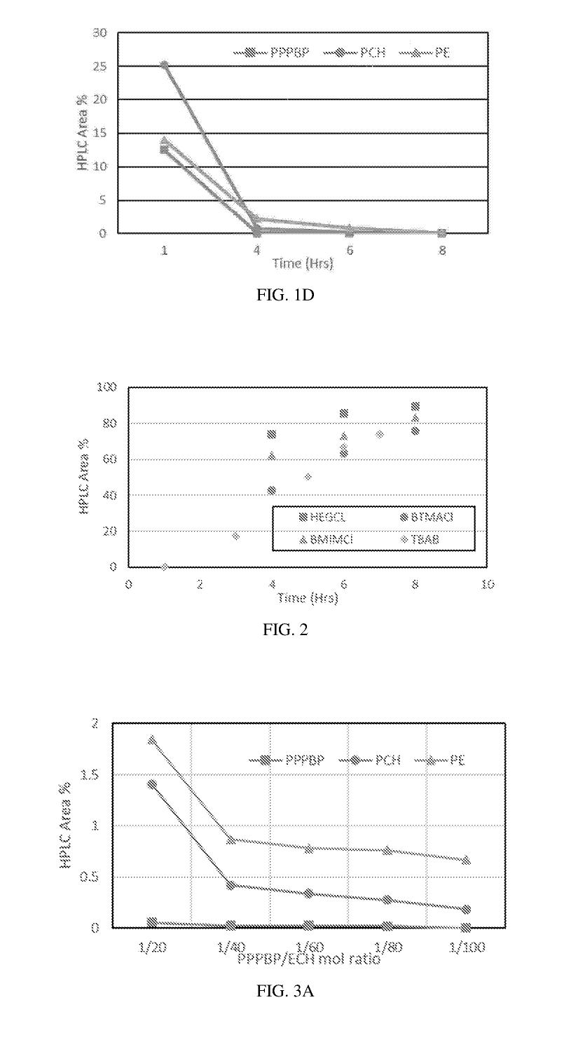

The effectiveness of each catalyst can be assessed from the results shown in FIGS. 1A, 1B, 1C, and 1D. Based on the results, it can be seen that HEGCl and BMIMC1 act as an effective catalyst with faster kinetics. TBAB and BTMAC1 act similarly with respect to reactivity of the phenolic OH with the ECH. Formation of the as-synthesized product was also monitored using HPLC. The product peak corresponding to the PPPBP-DGE was identified in the HPLC and the formation of the same was tracked. FIG. 2 shows the formation of epoxy with time. HEGCl showed faster kinetics compared to other catalysts in Process B.

### Example 8. PPPBP/Epichlorohydrin Ratio—Process A

Epoxidation of bisphenols is generally carried out using excess concentration of epichlorohydrin in order to overcome loss due to reaction with aqueous alkali and also to minimize generation of oligomeric products. The ratio of PPPBP/ECH is typically maintained at 1/20. This stoichiometry results in formation of diglycidyl ether of PPPBP with purity about 93 to 95% (about 3 to 4% oligomers, dimers, and other compounds).

In order to lower the oligomer content, the PPPBP/ECH ratio was increased while the remaining reaction parameters were kept constant. Process A was followed for these reactions. Results of product purity are shown in Table 4.

TABLE 4

| | Experiment No. | | | | |
|---|---|---|---|---|---|
| | 5a | 5b | 5c | 5d | 5e |
| PPPBP/ECH | 1/100 | 1/100 | 1/100 | 1/200 | 1/200 |
| Catalyst Type | BTMAC | BTMAC | TBAB | TBAB | BTMAC |
| Purity at the | 98.2 | 97.8 | 98.0 | 98.1 | 95.8 |
| end of reaction | | | | | |
| (HPLC area %) | | | | | |

### Example 9. PPPBP/Epichlorohydrin Ratio—PPPBP/Epichlorohydrin Ratio—Process B

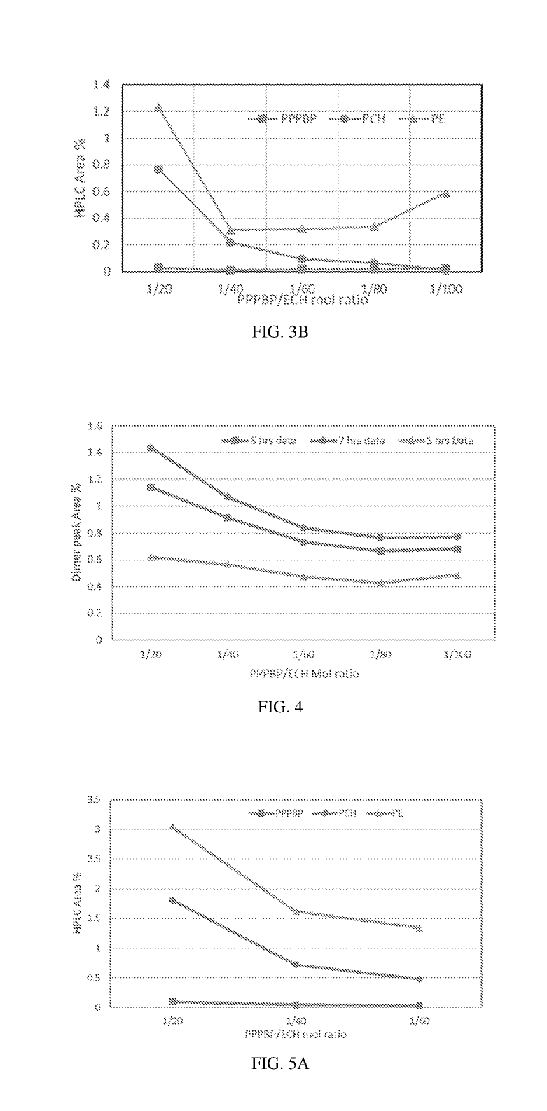

Further experiments were carried out using Process B to analyze the reaction between PPPBP and ECH. The reaction was carried out at 50° C. and 50 mol % catalyst concentration based on PPPBP, and the PPPBP/ECH mol ratio was varied from 1/20 to 1/100. Reaction aliquots were withdrawn over time and analyzed by HPLC to monitor reaction kinetics. Peaks corresponding to PPPBP, PCH, and PE were plotted for the samples at 6 hours and 7 hours. The results are shown in FIGS. 3A and 3B using TBAB catalyst.

As can be seen from FIGS. 3A and 3B, the residual concentration of PPPBP, PCH, and PE is relatively high for PPPBP/ECH ratio with low amount of ECH. However, the concentration of the residual starting material and intermediates remains constant from PPPBP/ECH molar ratio of 1/40 and 1/60.

The area percentage of the dimer peak was also analyzed. The results are shown in FIG. 4. FIG. 4 shows that dimer formation is greater at PPPBP/ECH ratio with low amount of ECH and significantly increases with time. However, the dimer area percentage remains constant for reaction with PPPBP/ECH 1/60 mol ratio and greater. Based on the analysis, 1/60 mol ratio is a useful mol ratio for the chlorohydrin formation reaction using TBAB catalyst.

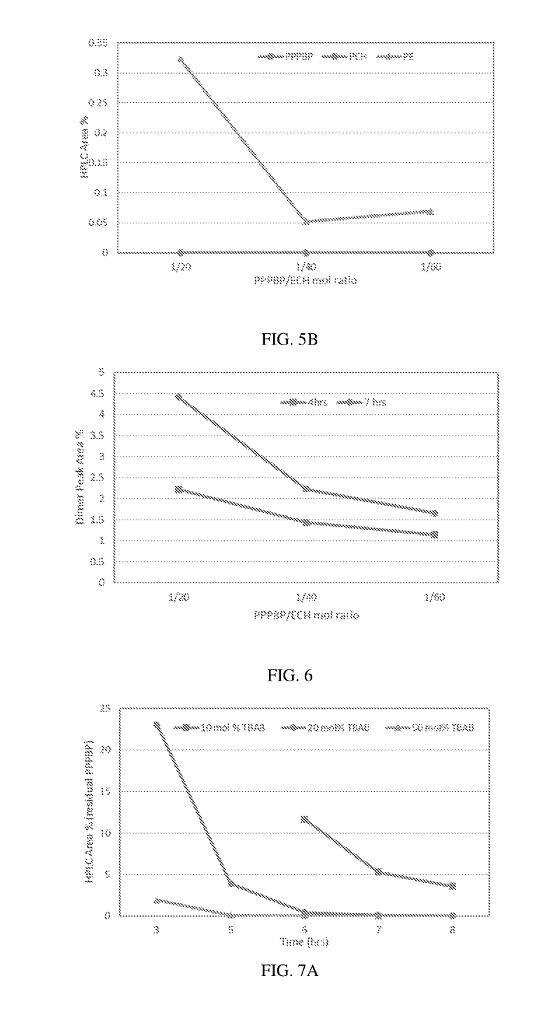

The analysis was repeated using HEGCl as catalyst at 50 mole % based on PPPBP. The PPPBP/ECH mol ratio was varied from 1/20 to 1/60. Peaks corresponding to PPPBP, PCH, and PE were plotted for the samples at 4 hours and 7 hours. Results are shown in FIGS. 5A and 5B.

The formation of dimer with time was also followed for these reactions using the same HPLC technique. Results are shown in FIG. 6.

FIG. 5A, 5B, and FIG. 6 show that 1/40 is a useful PPPBP/ECH mole ratio for the reaction carried out with HEGCl at a concentration of 50 mole %, based on PPPBP. The residual starting material (PPPBP) and intermediates (PCH and PE) were present in an insignificant amount at the 4th hour and almost exhausted by 7th hour. FIG. 6 shows the dimer concentration was relatively low for the reaction with mol ratio of 1/40 and above.

### Example 10. Catalyst Content—Process B

Catalyst content was studied by carrying out the chlorohydrin formation at various catalyst concentrations, ranging from 5 to 50 mol %. Initially TBAB was studied and then HEGCl was considered.

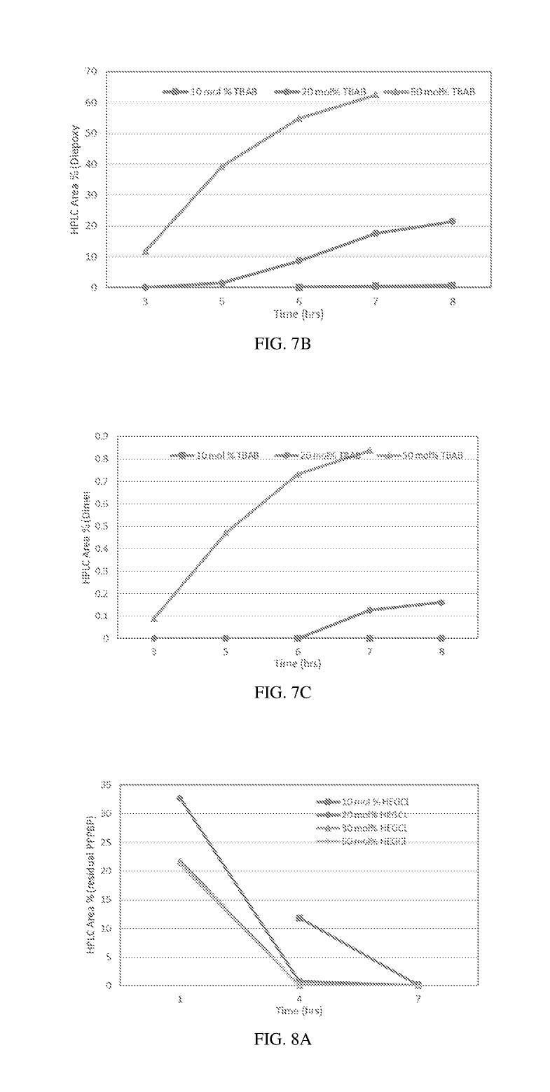

The reaction of PPPBP with ECH was carried out at 50° C. in presence of TBAB as catalyst. The catalyst concentration was maintained at 5, 10, 20, and 50 mole % based on moles of PPPBP. At 5 mol % of TBAB, PPPBP did not dissolve in the ECH for almost 16 hours. Therefore, the reaction was not monitored at this low level of TBAB. Residual PPPBP, PPPBP-DGE, and dimer concentration were monitored to determine a useful concentration of the catalyst. FIGS. 7A, 7B, and 7C show the effect of catalyst level on the HPLC area percent of PPPBP starting material (FIG. 7A), PPPBP-DGE product in the reaction mixture (FIG. 7B), and dimer side products (FIG. 7C).

As shown in FIGS. 7A, 7B, and 7C, the catalyst concentration has a strong effect on the rate of the reaction. This is indicated by higher rate of formation of PPPBP-DGE and also from the change in concentration of PPPBP starting material. At 50 mole % catalyst content, the reaction rate is appreciably higher than the reaction with 10 and 20 mole % catalyst. The catalyst concentration also has significant effect on the amount of dimer formed during the course of reaction. Higher catalyst concentration favors higher amount of dimer formation.

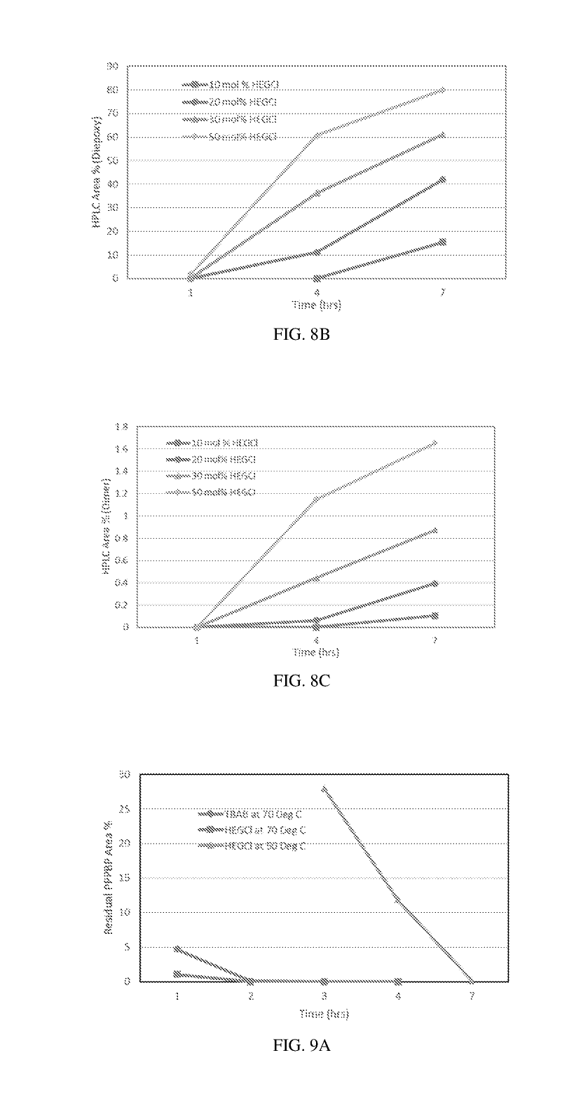

Similar conclusions can be drawn for the reactions where HEGCl was used as catalyst at different concentrations. These results are shown in FIGS. 8A, 8B, and 8C. These reactions appear to have higher rates even at lower catalyst concentration (at about 10 to 20 mole %) and dimer formation also occurred at a higher rate. However, 10 to 20 mol % seems to be a useful concentration for HEGCl under the given conditions.

### Example 11. Temperature Effect—Process B

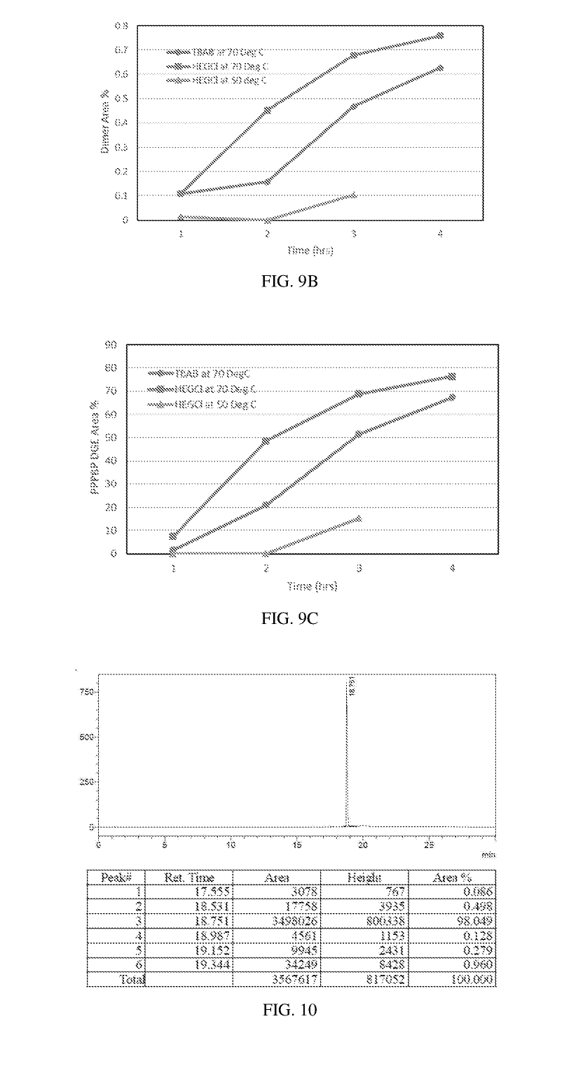

The effect of temperature was also studied for the chlorohydrin formation reaction. The PPPBP/ECH ratio was maintained at 1/60 and the catalyst level was either 10 mole % or 20 mol %. These reactions were also carried out at two different temperatures, 50° C. and 70° C. The results obtained are shown in FIGS. 9A, 9B, and 9C.

As shown in FIGS. 9A, 9B, and 9C, the reaction rate is appreciably higher at higher temperature, as is the rate of dimerization. In the presence of HEGCl at 70° C., almost all the PPPBP is converted to its intermediate within 2 to 3 hours of reaction, unlike the reaction with TBAB. The concentration of dimer formed at 70° C. is slightly high but still remains within manageable limits (<1%). In other words, step 1 of the process can be made faster by reacting at 70° C. with HEGCl as catalyst at 10 to 20 mol %.

### Example 12. Purity of PPPBP-DGE—Process B

The complete reaction was carried out. The mole ratio of PPPBP/ECH was maintained at 1/60, with HEGCl concentration of 10 mole % based on moles of PPPBP, and the reaction was carried out at 70° C. for 3 hours. After 3 hours, 50% NaOH (aq) solution (3 moles NaOH) was added dropwise for 1 hour and the reaction was allowed to progress for 3 hours. The final reaction aliquot was analyzed by HPLC. The purity of the final product was found to be 98.0%. The chromatogram of the final product in the reaction mixture is shown in FIG. 10.

### Example 13. Purity of PPPBP-DGE—Process C

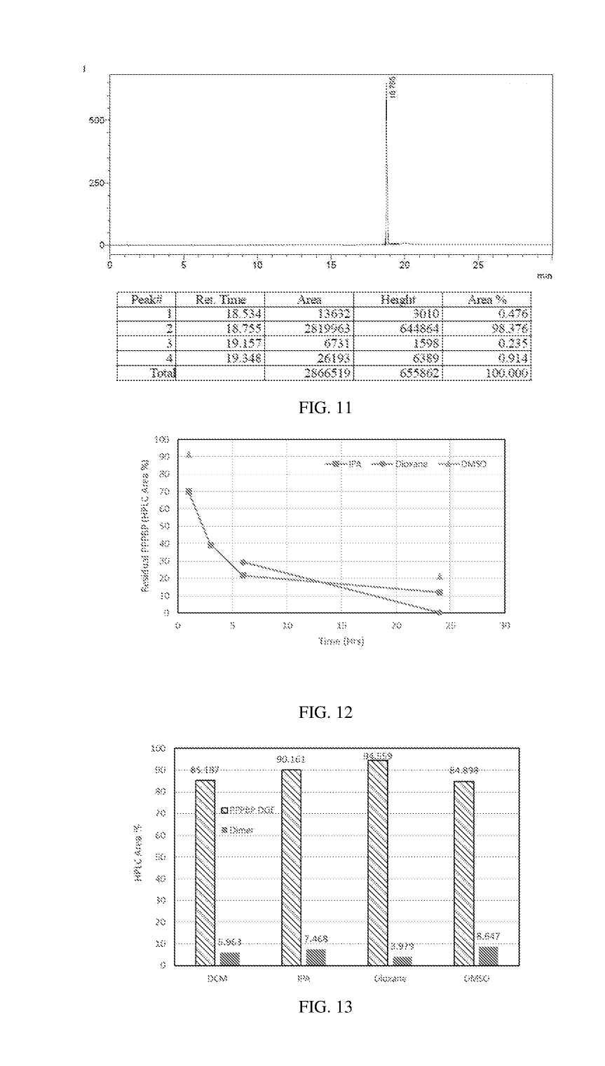

The reaction was also repeated with Process C. Once the first step of chlorohydrin formation reaction was complete (about 3 hrs), excess ECH was vacuum distilled. The residue was dissolved in DCM. NaOH solution with the same concentration (50% solution, 3 mol) was added slowly over period of 1 hour. The reaction continued for another 3 hours. The HPLC purity of the final product in the reaction mixture was found to be 98.36%. The HPLC chromatogram of the final product is shown in FIG. 11.

### Example 14. Process D

All the reactions carried out in presence of solvents were found to be very slow. In fact, when DCM and dioxane were used as solvent, PPPBP took a long time to dissolve. All the reactions continued for 24 hours and progress of the reaction was monitored using HPLC. FIG. 12 shows the progress of reaction in terms of disappearance of PPPBP with time. An appreciable amount of unreacted PPPBP remains in the reaction mixture even after 24 hours of reaction, especially when IPA and DMSO are used as solvents. After 24 hours of reaction in presence of different solvent, aqueous solution of NaOH (50%, 1:3 mol with respect to PPPBP) was added slowly and the reaction was further continued for 3 hours.

FIG. 13 shows the purity of the PPPBP-DGE (along with dimer) formed on addition of base (1:3 with respect to PPPBP) to the reaction mixture after 3 hours of reaction. This shows that under the given condition, a considerable amount of dimers is formed, resulting in drop of the overall purity of the final product.

### Example 15. Process E

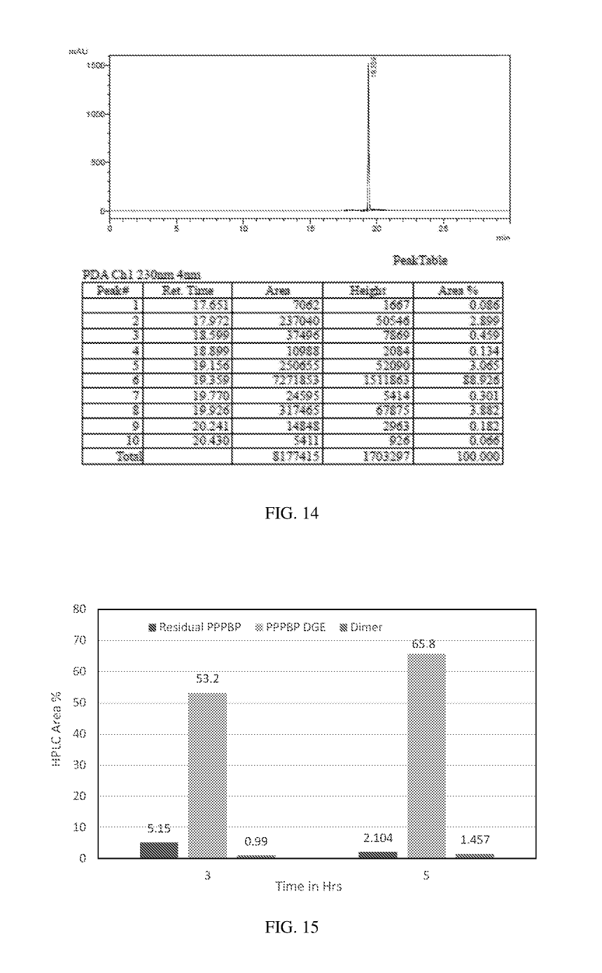

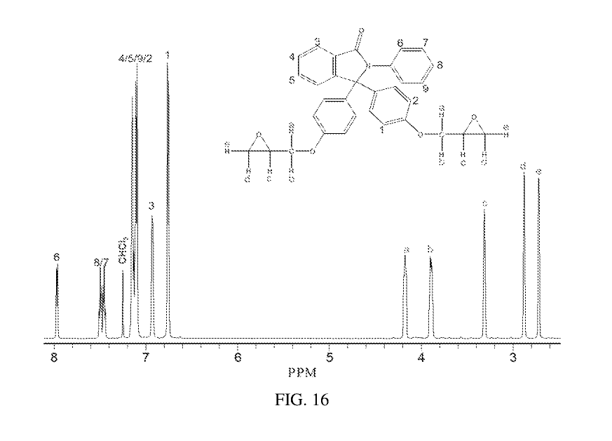

Using Process E, PPPBP solution was added slowly to flask containing ECH and catalyst solution. The dropwise mode of addition meant that PPPBP will always be in excess compared to ECH. Therefore, the process could be a way to avoid using large excess of ECH to form high purity end products. PPPBP solution in NaOH (20% solution, 3 mol per mole of PPPBP) was prepared and added dropwise for 4 hrs to a reaction mixture containing ECH (PPPBP/ECH mol ratio about 1/60) and HEGCl (10 mol %). The reaction was continued further for another 3 hrs with periodic monitoring using HPLC. The chromatogram of the final reaction mixture is shown in FIG. 14.

FIG. 14 shows that the purity of the final product is below 90% with dimer percentage close to 4%. A similar experiment was carried out with PPPBP solution in DMSO and when added to ECH/HEGCl solution at the same temperature, products with relatively low purity were formed. The reaction kinetics also slows down which can be seen from FIG. 15. Even after 5 hours of reaction at 70° C., ˜2% of PPPBP remains as unreacted and ˜1.45% of dimer already forms in the reaction mixture. This is considerably slower than the rate of conversion of reaction carried using either Process B or C.

The compositions, methods, articles and other aspects are further described by the Embodiments below.

### Embodiment 1

A process for preparing a diglycidyl ether of formula (1) to (9)

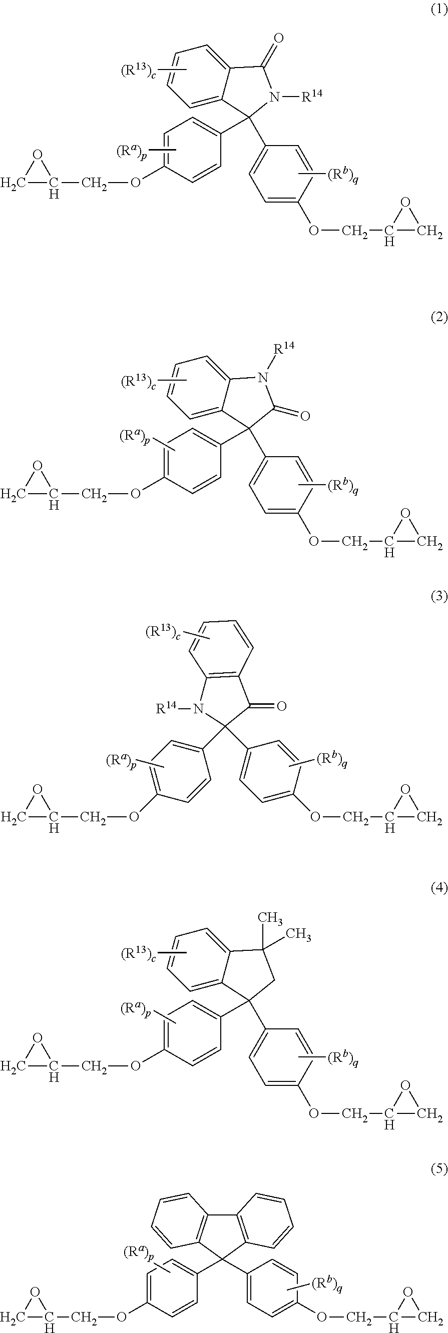

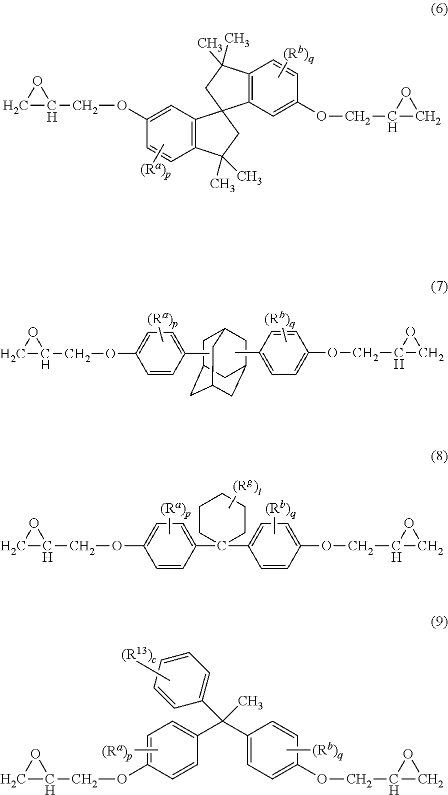

wherein
R^{a} and R^{b} at each occurrence are each independently halogen, C₁₋₁₂ alkyl, C₂₋₁₂ alkenyl, C₃₋₈ cycloalkyl, or C₁₋₁₂ alkoxy,
p and q at each occurrence are each independently 0 to 4,
R¹³ at each occurrence is independently halogen or C₁₋₆ alkyl,
c at each occurrence is independently 0 to 4,
R¹⁴ at each occurrence is independently C₁₋₆ alkyl, phenyl, or phenyl substituted with up to five halogens or C₁₋₆ alkyls,
R^{g} at each occurrence is independently halogen or C₁₋₁₂ alkyl, or two R^{g} groups together with the carbon atoms to which they are attached form a four-, five, or six-membered cycloalkyl group, and
t is 0 to 10;
   the process comprising

reacting a bisphenol of formula (1-b) to (9-b)

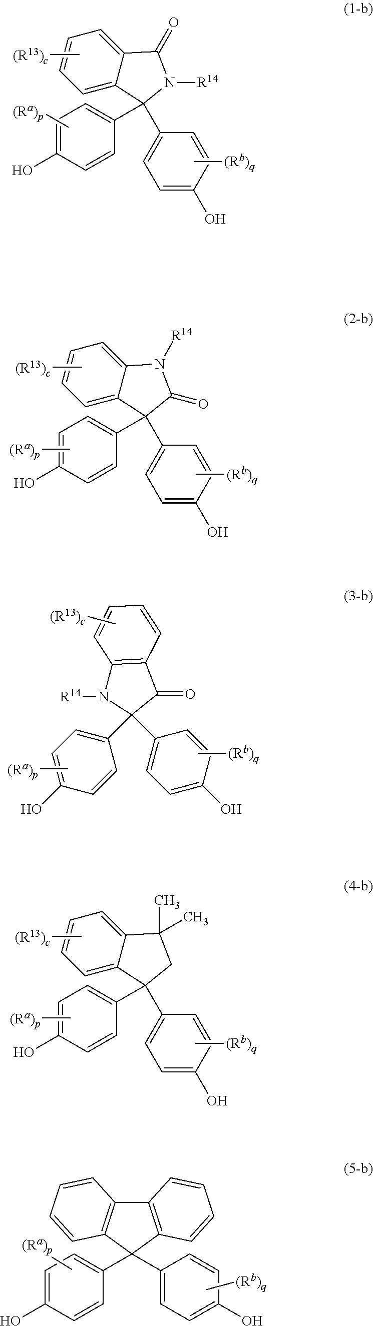

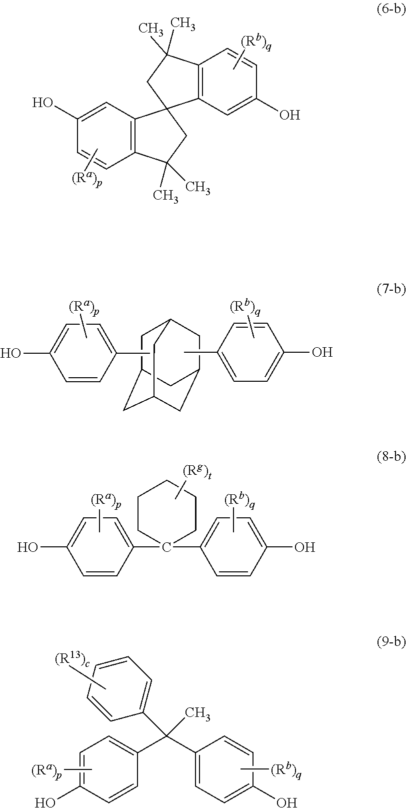

wherein R^{a}, R^{b}, p, q, R¹³, R¹⁴, c, R^{g}, and t are as defined in formulas (1) to (9), with epichlorohydrin and a catalyst, for a time and at a temperature to provide a first reaction mixture comprising a di-chlorohydrin of formula (1-d) to (9-d):

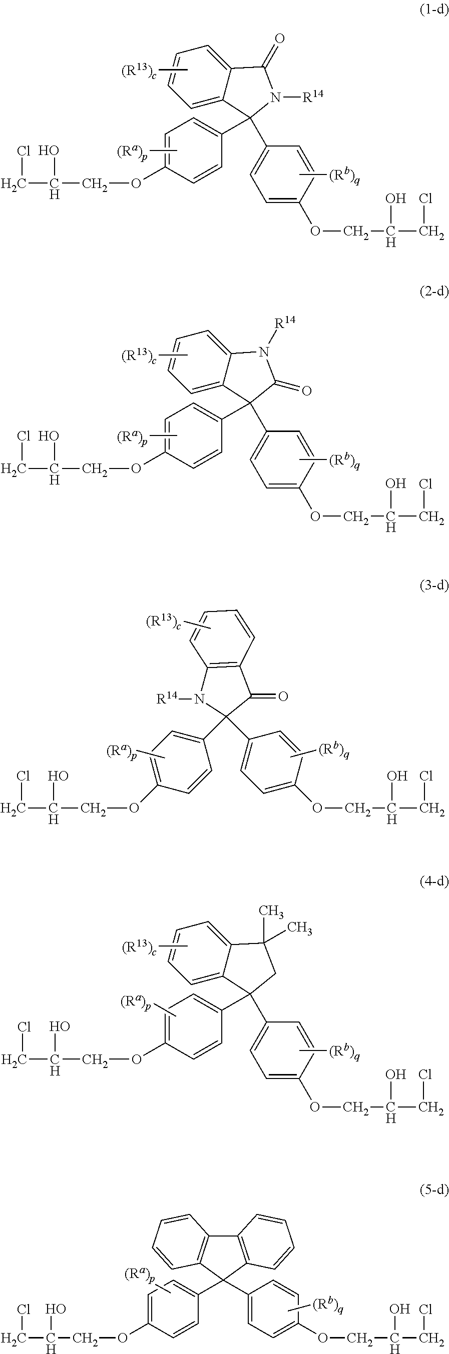

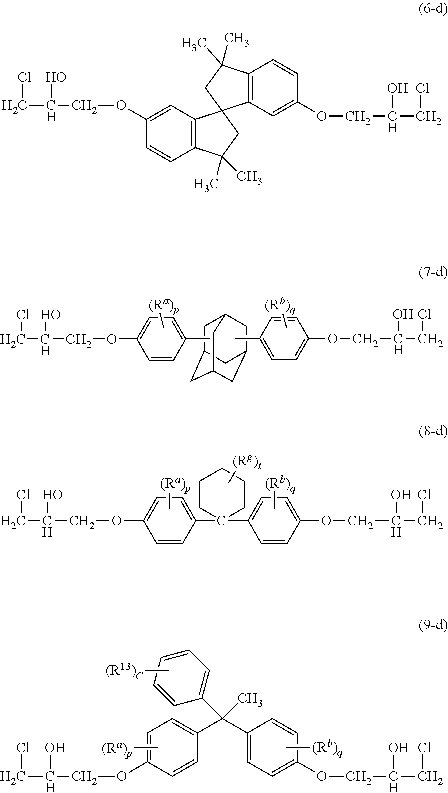

wherein R^{a}, R^{b}, p, q, R¹³, R¹⁴, c, R^{g}, and t are as defined in formulas (1) to (9);

adding a base to the first reaction mixture to provide a second reaction mixture over a period of 15 minutes to 3 hours; and

agitating the second reaction mixture for 2 to 5 hours at 40 to 60° C., to provide an as-synthesized diglycidyl ether of formula (1) to (9) having a purity of 96 to 99% or greater as determined by high performance liquid chromatography.

### Embodiment 2

The process of Embodiment 1, wherein the first reaction mixture further comprises a mono-substituted chlorohydrin of formula (1-e) to (9-e),

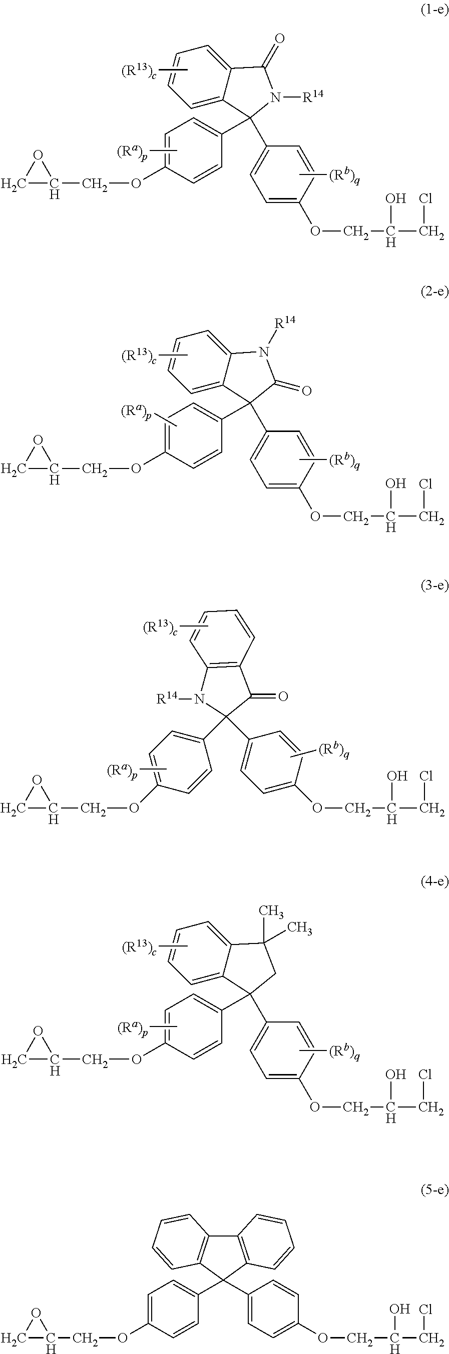

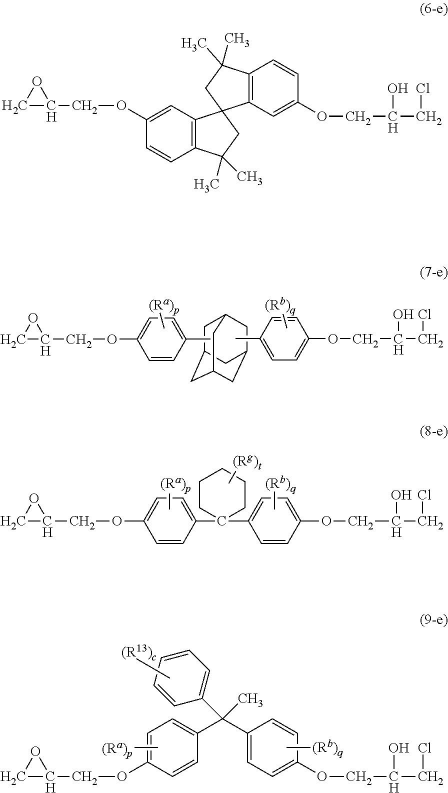

wherein R^{a}, R^{b}, p, q, R¹³, R¹⁴, c, R^{g}, and t are as defined in formulas (1) to (9), and

a diglycidyl ether of formula (1) to (9).

### Embodiment 3

The process of any one or more of Embodiments 1 to 2, wherein the bisphenol of formula (1-b) to (9-b) has a purity of 90 to 99.9%, or 92 to 99.9%, or 95 to 99.9%, or 97 to 99.9.

### Embodiment 4

The process of any one or more of Embodiments 1 to 3, wherein the molar ratio of the bisphenol of formula (1-b) to (9-b)/epichlorohydrin is 1/20 to 1/80, preferably 1/35 to 1/65.

### Embodiment 5

The process of any one or more of Embodiments 1 to 4, wherein the catalyst comprises a quaternary ammonium salt, quaternary phosphonium salt, sulphoxide, sulpholane, guanidinium salt, imidazolium salt, or a combination comprising at least one of the foregoing.

### Embodiment 6

The process of any one or more of Embodiments 1 to 5, wherein the catalyst is hexaethylguanidium chloride and the catalyst is present at 2 to 30 mole percent, based on the moles of the bisphenol of formula (1-b) to (9-b).

### Embodiment 7

The process of any one or more of Embodiments 1 to 5, wherein the catalyst is tetrabutylammonium bromide and the catalyst is present at 10 to 60 mole percent, based on the moles of bisphenol of formula (1-b) to (9-b).

### Embodiment 8

The process of any one or more of Embodiments 1 to 7, wherein the reacting the bisphenol of formula (1-b) to (9-b) and the epichlorohydrin in the presence of the catalyst is for 2 to 9 hours at 50 to 80° C., preferably 2.5 to 7 hours at 50 to 75° C.

### Embodiment 9

The process of any one or more of Embodiments 1 to 8, wherein the base is sodium hydroxide or potassium hydroxide, and the adding is over 30 minutes to 3 hours; and wherein the agitating the second reaction mixture is for 3 to 4 hours at 40 to 60° C., preferably at 45 to 55° C.

### Embodiment 10

The process of any one or more of Embodiments 1 to 9, further comprising removing unreacted epichlorohydrin from the first reaction mixture, preferably by distilling.

### Embodiment 11

The process of any one or more of Embodiments 1 to 10, wherein the as-synthesized diglycidyl ether of formula (1) to (9) has at least one of

a purity of 97% or greater, as determined by high performance liquid chromatography;

a hydrolyzable chloride content of less than 2,000, less than 1,000, or less than 800 ppm;

an epoxy equivalent weight within 4%, or within 2%, or within 1%, or within 0.5% of the epoxy equivalent weight of the pure compound;

less than 1 wt % of the corresponding dimer, as measured by high performance liquid chromatography;

less than 50, or less than 40, or less than 20, or less than 10, or less than 5 ppm of residual epichlorohydrin;

less than 1 total wt % of the di-chlorohydrin of formula (1-d) to (9-d) and the mono-substituted chlorohydrin of formula (1-e) to (9-e), as determined by HPLC; or

a residual moisture content of 0.01 to 0.5 wt %, or 0.1 to 0.4 wt %.

### Embodiment 12

The process of any one or more of Embodiments 1 to 6 or 8 to 11, wherein

the molar ratio of the bisphenol of formula (1-b) to (9-b)/epichlorohydrin is 1/30 to 1/70,

the catalyst is hexaethylguanidium chloride and the catalyst is present in an amount of 5 to 50 mol % based on the bisphenol of formula (1-b) to (9-b),

the agitating is carried out for 3 to 4 hours at 40 to 60° C., preferably at 45 to 55° C., and

the as-synthesized diglycidyl ether of formula (1) to (9) has a purity of 97% or greater as determined by high performance liquid chromatography, without performing a chromatographic purification.

### Embodiment 13

The process of any one or more of Embodiments 1 to 12, further comprising isolating the as-synthesized diglycidyl ether of formula (1) to (9) to provide an isolated diglycidyl ether of formula (1) to (9) having has at least one of

a purity of 97% or greater, as determined by high performance liquid chromatography;

a hydrolyzable chloride content of less than 2,000, less than 1,000, or less than 800 ppm;

an epoxy equivalent weight within 4%, or within 2%, or within 1%, or within 0.5% of the epoxy equivalent weight of the pure compound;

less than 1 wt % of the corresponding dimer, as measured by high performance liquid chromatography;

less than 50, or less than 40, or less than 20, or less than 10, or less than 5 ppm of residual epichlorohydrin;

less than 1 total wt % of the di-chlorohydrin of formula (1-d) to (9-d) and the mono-substituted chlorohydrin of formula (1-e) to (9-e), as determined by HPLC; or

a residual moisture content of 0.01 to 0.5 wt %, or 0.1 to 0.4 wt %.

### Embodiment 14

The process of any one or more of Embodiments 1 to 13, wherein the diglycidyl ether of formula (1) to (9) is of formula (1-a), (2-a), (3-a), (4-a), (5-a), (6-a), (7-a), (8-a), (8-a′), (8-a″), or (9-a).

### Embodiment 15

A process for preparing a diglycidyl ether of formula (1-a)

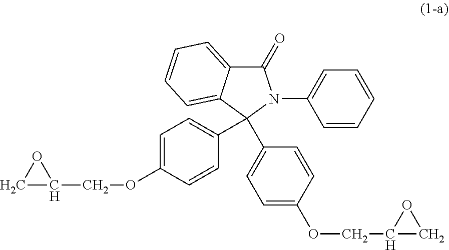 [SMILES: O=C1c2ccccc2C(c2ccc(OCC3CO3)cc2)(c2ccc(OCC3CO3)cc2)N1c1ccccc1]

the process comprising

reacting epichlorohydrin and a bisphenol of formula (1-c)

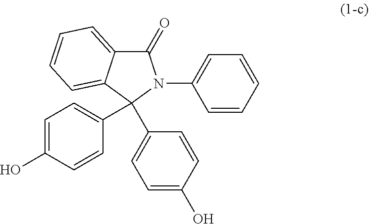 [SMILES: O=C1c2ccccc2C(c2ccc(O)cc2)(c2ccc(O)cc2)N1c1ccccc1]

in the presence of hexaethylguanidium chloride in an amount of 5 to 50 mol % based on the compound of formula (1-c), for 3 to 5 hours at 60 to 80° C., wherein the molar ratio of (compound of formula (1-c)/epichlorohydrin) is 1/30 to 1/70, to form a first reaction mixture comprising the chlorohydrin of formula (1-g)

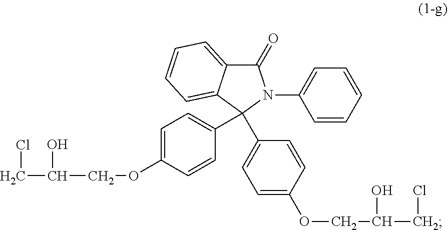 [SMILES: O=C1c2ccccc2C(c2ccc(OCC(O)CCl)cc2)(c2ccc(OCC(O)CCl)cc2)N1c1ccccc1]

distilling unreacted epichlorohydrin from the first reaction mixture to form a solid residue;

dissolving the solid residue in a solvent;

adding sodium hydroxide or potassium hydroxide to the dissolved residue to provide a second reaction mixture; and

agitating the second reaction mixture for 3 to 4 hours at 40 to 60° C., to provide an as-synthesized diglycidyl ether of formula (1-a) having at least one of
a purity of 97% or greater, as determined by high performance liquid chromatography;

a hydrolyzable chloride content of less than 2,000, less than 1,000, or less than 800 ppm;

an epoxy equivalent weight within 4%, or within 2%, or within 1%, or within 0.5% of the epoxy equivalent weight of the pure compound;

less than 1 wt % of the corresponding dimer, as measured by high performance liquid chromatography;

less than 50, or less than 40, or less than 20, or less than 10, or less than 5 ppm of residual epichlorohydrin;

less than 1 total wt % of the di-chlorohydrin of formula (1-d) to (9-d) and the mono-substituted chlorohydrin of formula (1-e) to (9-e), as determined by HPLC; or

a residual moisture content of 0.01 to 0.5 wt %, or 0.1 to 0.4 wt %.

### Embodiment 16

The process of Embodiment 15, wherein the first reaction mixture further comprises a monochlorohydrin of formula (1-f):

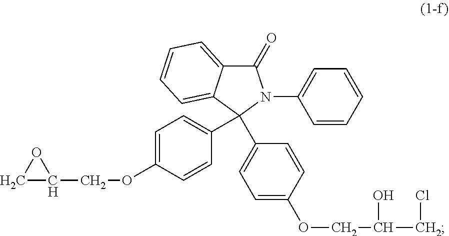 [SMILES: O=C1c2ccccc2C(c2ccc(OCC(O)CCl)cc2)(c2ccc(OCC3CO3)cc2)N1c1ccccc1]

the diglycidyl ether of formula (1-a).

### Embodiment 17

An as-synthesized or isolated diglycidyl ether of formula (1) to (9) made by the process of any one or more of Embodiment 1 to 16.

### Embodiment 18

A diglycidyl ether of formula (1) to (9), wherein the diglycidyl ether has less than 1 total wt % of the di-chlorohydrin of formula (1-d) to (9-d) and the mono-substituted chlorohydrin of formula (1-e) to (9-e), as determined by HPLC; and one or more of:

a purity of 97% or greater, as determined by high performance liquid chromatography;

a hydrolyzable chloride content of less than 2,000, less than 1,000, or less than 800 ppm;

an epoxy equivalent weight within 4%, or within 2%, or within 1%, or within 0.5% of the epoxy equivalent weight of the pure compound;

less than 1 wt % of the corresponding dimer, as measured by high performance liquid chromatography;

less than 50, or less than 40, or less than 20, or less than 10, or less than 5 ppm of residual epichlorohydrin; or

a residual moisture content of 0.01 to 0.5 wt %, or 0.1 to 0.4 wt %.

### Embodiment 19

A cured composition comprising the cured product of the diglycidyl ether of Embodiment 17 or 18, and an article comprising the same.

The compositions, methods, and articles can alternatively comprise, consist of, or consist essentially of, any appropriate components or steps herein disclosed. The compositions, methods, and articles can additionally, or alternatively, be formulated so as to be devoid, or substantially free, of any steps, components, materials, ingredients, adjuvants, or species that are otherwise not necessary to the achievement of the function or objectives of the compositions, methods, and articles.

The singular forms “a,” “an,” and “the” include plural referents unless the context clearly dictates otherwise. “Or” means “and/or” unless clearly indicated otherwise by context.

The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. Unless defined otherwise, technical and scientific terms used herein have the same meaning as is commonly understood by one of skill in the art to which this disclosure belongs. A “combination” is inclusive of blends, mixtures, alloys, reaction products, and the like.

As used herein, the term “hydrocarbyl” and “hydrocarbon” refers broadly to a substituent comprising carbon and hydrogen, optionally with 1 to 3 heteroatoms, for example, oxygen, nitrogen, halogen, silicon, sulfur, or a combination thereof; “alkyl” refers to a straight or branched chain, saturated monovalent hydrocarbon group; “alkylene” refers to a straight or branched chain, saturated, divalent hydrocarbon group; “alkylidene” refers to a straight or branched chain, saturated divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicyclic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents or variables are permissible provided that the substitutions do not significantly adversely affect synthesis or use of the compound. Exemplary groups that can be present on a “substituted” position include, but are not limited to, cyano; hydroxyl; nitro; azido; alkanoyl (such as a C₂₋₆ alkanoyl group such as acyl); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxys; C₆₋₁₀ aryloxy such as phenoxy; C₁₋₆ alkylthio; C₁₋₆ or C₁₋₃ alkylsulfinyl; C₁₋₆ or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 1 to 3 separate or fused rings and from 6 to 18 ring carbon atoms, with benzyloxy being an exemplary arylalkoxy.

All cited patents, patent applications, and other references are incorporated herein by reference in their entirety. However, if a term in the present application contradicts or conflicts with a term in the incorporated reference, the term from the present application takes precedence over the conflicting term from the incorporated reference. Unless otherwise specified, the test methods used are those current as of the filing date of the priority provisional application.

While typical embodiments have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

1. A process for preparing a diglycidyl ether of formula (1) to (9) 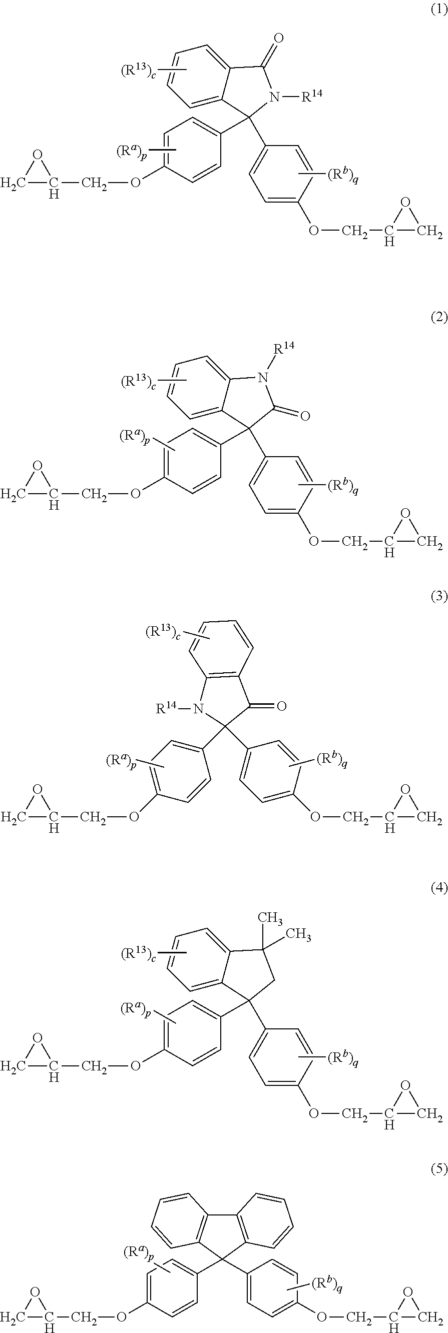 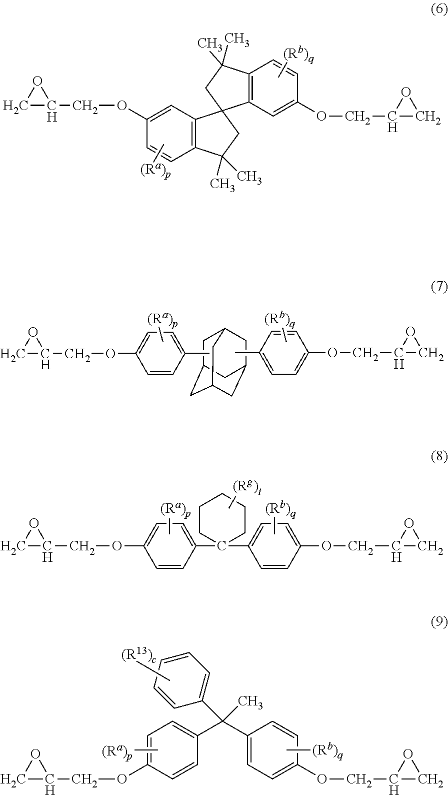
wherein
R^{a} and R^{b} at each occurrence are each independently halogen, C₁₋₁₂ alkyl, C₂₋₁₂ alkenyl, C₃₋₈ cycloalkyl, or C₁₋₁₂ alkoxy,
p and q at each occurrence are each independently 0 to 4,
R¹³ at each occurrence is independently halogen or C₁₋₆ alkyl,
c at each occurrence is independently 0 to 4,
R¹⁴ at each occurrence is independently C₁₋₆ alkyl, phenyl, or phenyl substituted with up to five halogens or C₁₋₆ alkyls,
R^{g} at each occurrence is independently halogen or C₁₋₁₂ alkyl, or two R^{g} groups together with the carbon atoms to which they are attached form a four-, five, or six-membered cycloalkyl group, and
t is 0 to 10;
the process comprising
reacting a bisphenol of formula (1-b) to (9-b) 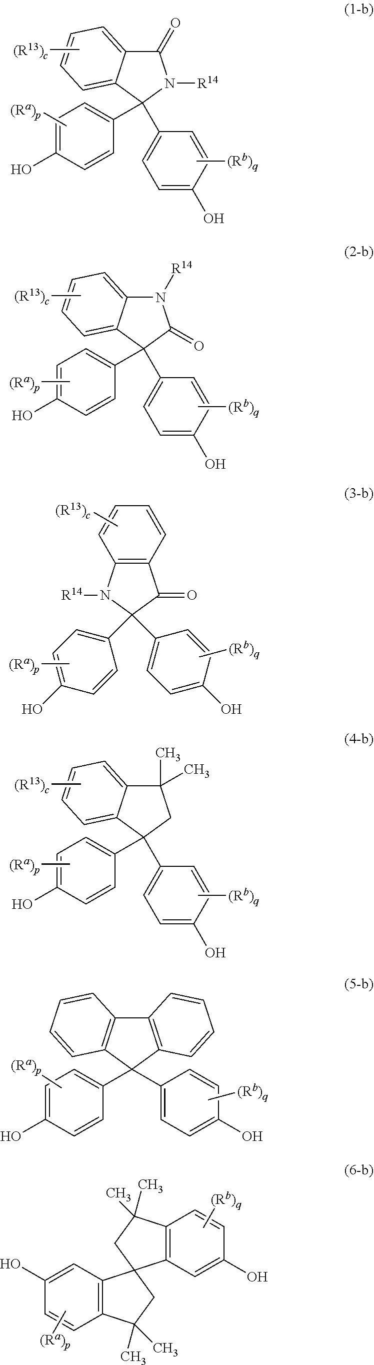 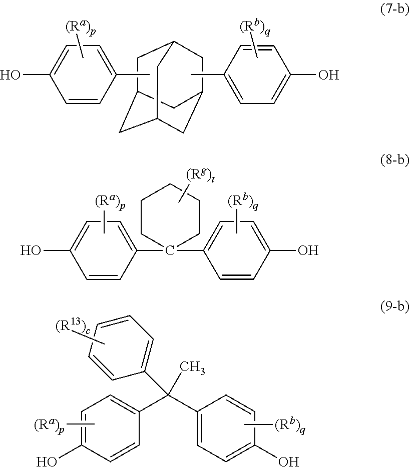
wherein R^{a}, R^{b}, p, q, R¹³, R¹⁴, c, R^{g}, and t are as defined in formulas (1) to (9), with epichlorohydrin and a catalyst, for a time and at a temperature to provide a first reaction mixture comprising a di-chlorohydrin of formula (1-d) to (9-d): 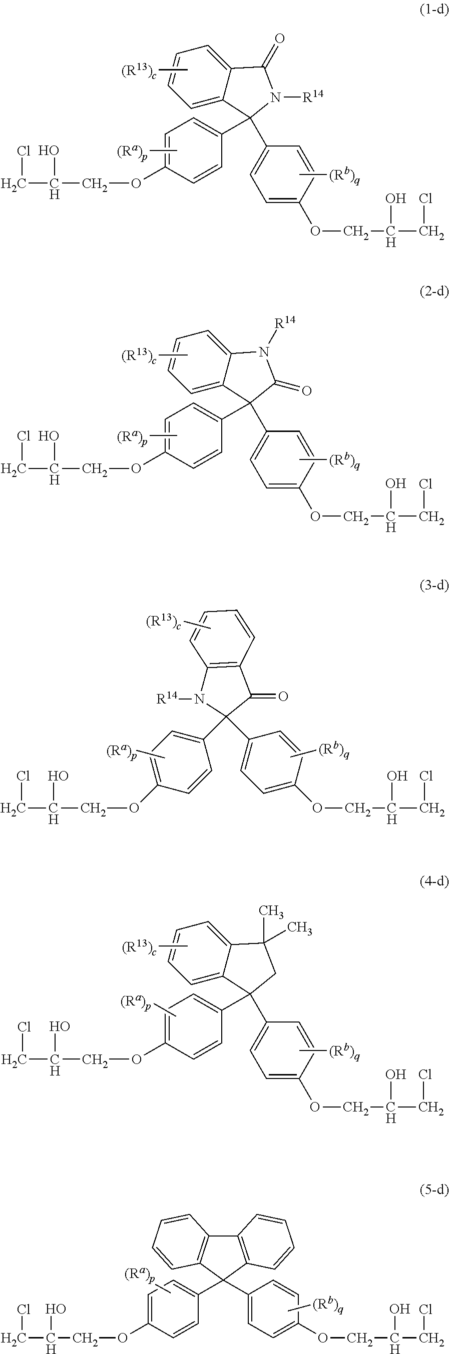 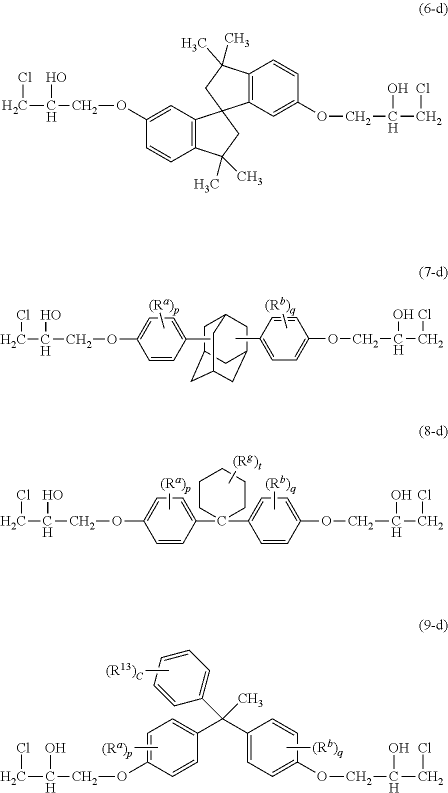
wherein R^{a}, R^{b}, p, q, R¹³, R¹⁴, c, R^{g}, and t are as defined in formulas (1) to (9);
adding a base to the first reaction mixture to provide a second reaction mixture over a period of 15 minutes to 3 hours; and
agitating the second reaction mixture for 2 to 5 hours at 40 to 60° C., to provide an as-synthesized diglycidyl ether of formula (1) to (9) having a purity of 96 to 99% or greater as determined by high performance liquid chromatography.

2. The process of claim 1, wherein the first reaction mixture further comprises a mono-substituted chlorohydrin of formula (1-e) to (9-e), 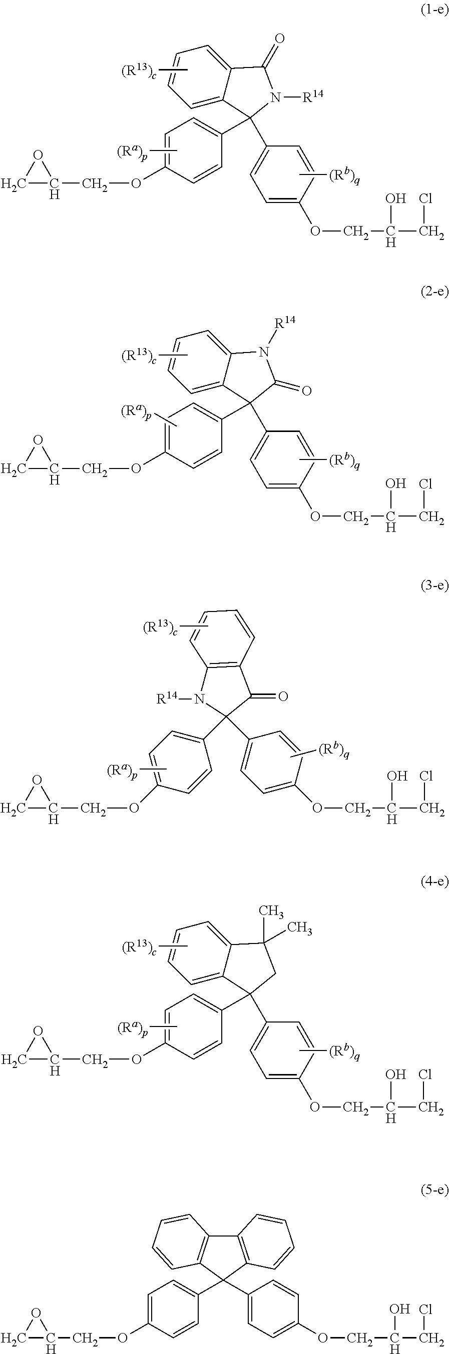 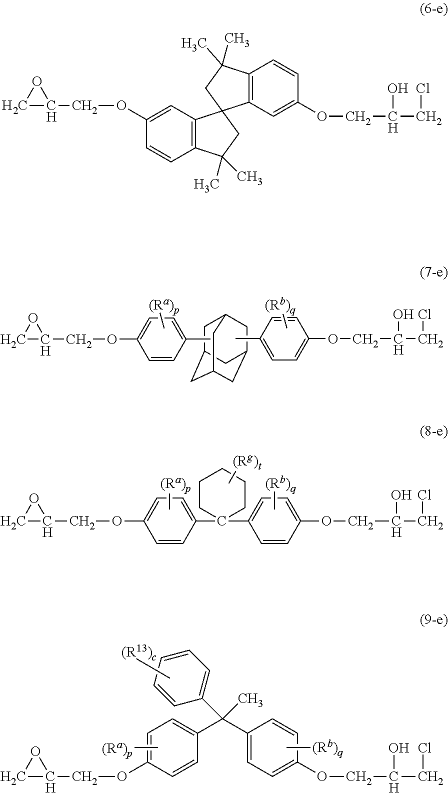
wherein R^{a}, R^{b}, p, q, R¹³, R¹⁴, c, R^{g}, and t are as defined in formulas (1) to (9), and
a diglycidyl ether of formula (1) to (9).

3. The process of claim 1, wherein the bisphenol of formula (1-b) to (9-b) has a purity of 90 to 99.9%.

4. The process of claim 1, wherein the molar ratio of the bisphenol of formula (1-b) to (9-b)/epichlorohydrin is 1/20 to 1/80.

5. The process of claim 1, wherein the catalyst comprises a quaternary ammonium salt, quaternary phosphonium salt, sulphoxide, sulpholane, guanidinium salt, imidazolium salt, or a combination comprising at least one of the foregoing.

6. The process of claim 1, wherein the catalyst is hexaethylguanidium chloride and the catalyst is present at 2 to 30 mole percent, based on the moles of the bisphenol of formula (1-b) to (9-b).

7. The process of claim 1, wherein the catalyst is tetrabutylammonium bromide and the catalyst is present at 10 to 60 mole percent, based on the moles of bisphenol of formula (1-b) to (9-b).

8. The process of claim 1, wherein the reacting the bisphenol of formula (1-b) to (9-b) and the epichlorohydrin in the presence of the catalyst is for 2 to 9 hours at 50 to 80° C.

9. The process of claim 1, wherein the base is sodium hydroxide or potassium hydroxide, and the adding is over 30 minutes to 3 hours; and wherein the agitating the second reaction mixture is for 3 to 4 hours at 40 to 60° C.

10. The process of claim 1, further comprising removing unreacted epichlorohydrin from the first reaction mixture.

11. The process of claim 1, wherein the as-synthesized diglycidyl ether of formula (1) to (9) has at least one of
a purity of 97% or greater, as determined by high performance liquid chromatography;
a hydrolyzable chloride content of less than 2,000 ppm;
an epoxy equivalent weight within 4% of the epoxy equivalent weight of the pure compound;
less than 1 wt % of the corresponding dimer, as measured by high performance liquid chromatography;
less than 50 ppm of residual epichlorohydrin;
less than 1 total wt % of the di-chlorohydrin of formula (1-d) to (9-d) and the mono-substituted chlorohydrin of formula (1-e) to (9-e), as determined by HPLC; or
a residual moisture content of 0.01 to 0.5 wt %.

12. The process of claim 1, wherein
the molar ratio of the bisphenol of formula (1-b) to (9-b)/epichlorohydrin is 1/30 to 1/70,
the catalyst is hexaethylguanidium chloride and the catalyst is present in an amount of 5 to 50 mol % based on the bisphenol of formula (1-b) to (9-b),
the agitating is carried out for 3 to 4 hours at 40 to 60° C., and
the as-synthesized diglycidyl ether of formula (1) to (9) has a purity of 97% or greater as determined by high performance liquid chromatography, without performing a chromatographic purification.

13. The process of claim 1, further comprising isolating the as-synthesized diglycidyl ether of formula (1) to (9) to provide an isolated diglycidyl ether of formula (1) to (9) having has at least one of
a purity of 97% or greater, as determined by high performance liquid chromatography;
a hydrolyzable chloride content of less than 2,000 ppm;
an epoxy equivalent weight within 4% of the epoxy equivalent weight of the pure compound;
less than 1 wt % of the corresponding dimer, as measured by high performance liquid chromatography;
less than 50 ppm of residual epichlorohydrin;
less than 1 total wt % of the di-chlorohydrin of formula (1-d) to (9-d) and the mono-substituted chlorohydrin of formula (1-e) to (9-e), as determined by HPLC; or
a residual moisture content of 0.01 to 0.5 wt %.

14. The process of claim 1, wherein the diglycidyl ether of formula (1) to (9) is of formula (1-a), (2-a), (3-a), (4-a), (5-a), (6-a), (7-a), (8-a), (8-a′), (8-a″), or (9-a).

15. A process for preparing a diglycidyl ether of formula (1-a) 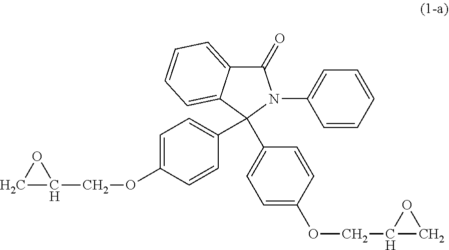 [SMILES: O=C1c2ccccc2C(c2ccc(OCC3CO3)cc2)(c2ccc(OCC3CO3)cc2)N1c1ccccc1]
the process comprising
reacting epichlorohydrin and a bisphenol of formula (1-c) 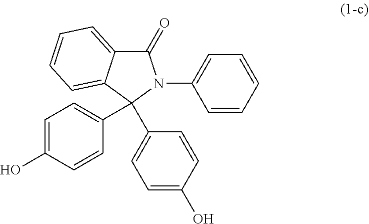 [SMILES: O=C1c2ccccc2C(c2ccc(O)cc2)(c2ccc(O)cc2)N1c1ccccc1]
in the presence of hexaethylguanidium chloride in an amount of 5 to 50 mol % based on the compound of formula (1-c), for 3 to 5 hours at 60 to 80° C., wherein the molar ratio of compound of formula (1-c)/epichlorohydrin is 1/30 to 1/70, to form a first reaction mixture comprising a chlorohydrin of formula (1-g) 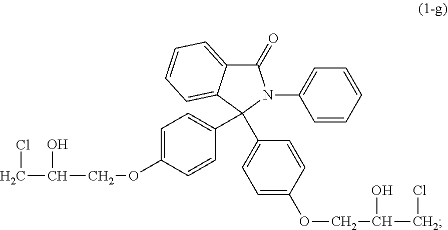 [SMILES: O=C1c2ccccc2C(c2ccc(OCC(O)CCl)cc2)(c2ccc(OCC(O)CCl)cc2)N1c1ccccc1]
distilling unreacted epichlorohydrin from the first reaction mixture to form a solid residue;
dissolving the solid residue in a solvent;
adding sodium hydroxide or potassium hydroxide to the dissolved residue to provide a second reaction mixture; and
agitating the second reaction mixture for 3 to 4 hours at 40 to 60° C., to provide an as-synthesized diglycidyl ether of formula (1-a) having at least one of
a purity of 97% or greater, as determined by high performance liquid chromatography;
a hydrolyzable chloride content of less than 2,000 ppm;
an epoxy equivalent weight within 4% of the epoxy equivalent weight of the pure compound;
less than 1 wt % of the corresponding dimer, as measured by high performance liquid chromatography;
less than 50 ppm of residual epichlorohydrin;
less than 1 total wt % of the di-chlorohydrin of formula (1-d) to (9-d) and the mono-substituted chlorohydrin of formula (1-e) to (9-e), as determined by HPLC; or
a residual moisture content of 0.01 to 0.5 wt %.

16. The process of claim 15, wherein the first reaction mixture further comprises a monochlorohydrin of formula (1-f): 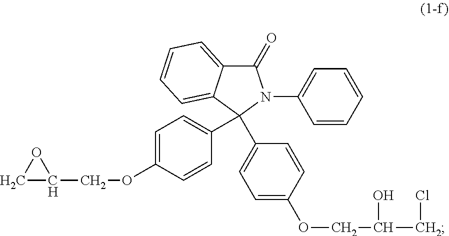 [SMILES: O=C1c2ccccc2C(c2ccc(OCC(O)CCl)cc2)(c2ccc(OCC3CO3)cc2)N1c1ccccc1]
and
the diglycidyl ether of formula (1-a).

17. An as-synthesized or isolated diglycidyl ether of formula (1) to (9) made by the process of claim 1.

18. The as-synthesized or isolated diglycidyl ether of claim 17, wherein the diglycidyl ether has less than 1 total wt % of the di-chlorohydrin of formula (1-d) to (9-d) and the mono-substituted chlorohydrin of formula (1-e) to (9-e), as determined by HPLC; and one or more of:
a purity of 97% or greater, as determined by high performance liquid chromatography;
a hydrolyzable chloride content of less than 2,000 ppm;
an epoxy equivalent weight within 4% of the epoxy equivalent weight of the pure compound;
less than 1 wt % of the corresponding dimer, as measured by high performance liquid chromatography;
less than 50 ppm of residual epichlorohydrin; or
a residual moisture content of 0.01 to 0.5 wt %.

19. The process of claim 1, further comprising curing the diglycidyl ether.

20. The process of claim 15, further comprising curing the diglycidyl ether.
